(19) United States
(12) Patent Application Publication
(10) Pub. No.: US 2024/0382582 A1
(43) Pub. Date: Nov. 21, 2024

(54) Immunomodulatory proteins- or targeting proteins-expressing nanovesicles, methods of preparing the nanovesicle and use thereof

(71) Applicant: POSTECH RESEARCH AND BUSINESS DEVELOPMENT FOUNDATION, Pohang-si (KR)

(72) Inventors: Jae Sung PARK, Pohang-si (KR); Su Yeon KIM, Pohang-si (KR); Seung Ik OH, Pohang-si (KR); Chungmin HAN, Pohang-si (KR)

(21) Appl. No.: 18/663,339

(22) Filed: May 14, 2024

(30) Foreign Application Priority Data
May 15, 2023 (KR) 10-2023-0062223
May 15, 2023 (KR) 10-2023-0062224
May 8, 2024 (KR) 10-2024-0060860
May 14, 2024 (KR) 10-2024-0063410

(51) Int. Cl. A61K 39/215; A61K 9/51; A61K 39/00; A61P 31/14; C12N 7/00

(52) U.S. Cl. CPC A61K 39/215; A61K 9/5176; A61K 9/5192; A61P 31/14; C12N 7/00; A61K 2039/55555; A61K 2039/575; C12N 2770/20023; C12N 2770/20034

(57) ABSTRACT

The present invention relates to nanovesicles which express immunomodulatory proteins or targeting proteins, methods for preparing the same and uses thereof. More specifically, the present invention provides plasma membrane bleb-based nanovesicles which are prepared more homogeneously than existing plasma membrane bleb-based nanovesicles, by using cell lines expressing various immunomodulatory proteins or targeting proteins in the plasma membrane as materials, methods for preparing the nanovesicles, pharmaceutical compositions including the nanovesicles, methods for inducing immunity using the nanovesicles and methods for signal transduction or targeting using the nanovesicles.

### REFERENCE TO AN ELECTRONIC SEQUENCE LISTING

The contents of the electronic sequence listing (EPL20246634US_SEQ.xml; Size: 7,448 bytes; and Date of Creation: May 14, 2024) is herein incorporated by reference in its entirety. The contents of the electronic sequence listing in no way introduces new matter into the specification.

### TECHNICAL FIELD

The present invention relates to nanovesicles which express immunomodulatory proteins or targeting proteins, methods for preparing the same and uses thereof. More specifically, the present invention provides plasma membrane bleb-based nanovesicles which are prepared more homogeneously than existing cell-engineered nanovesicles, by using cell lines expressing various immunomodulatory proteins or targeting proteins in the plasma membrane as materials, methods for preparing the nanovesicles, pharmaceutical compositions including the nanovesicles, methods for inducing immunity using the nanovesicles and methods for signal transduction or targeting using the nanovesicles.

### BACKGROUND ART

Protein delivery is being applied in various therapeutic applications, and technologies for injecting such proteins into the body are being developed, but they have limitations in terms of the duration and targeting of proteins in the body.

When only soluble proteins are injected into the body, the soluble proteins are rapidly decomposed and removed in the body due to the rapid half-life thereof, which may limit the therapeutic efficacy. In addition, according to Non-Patent Documents 1 and 2, since targeting is difficult when using soluble proteins, there is a possibility of toxicity due to overdose administration. Due to these limitations, alternative methods for protein delivery, such as increasing the half-life in the body using synthetic nanoparticles and antibody conjugation technology, are being studied recently.

Nanoparticles can increase the half-life in the body, thereby improving the pharmacokinetics of proteins, and can remain in the body for a relatively long period of time without being eliminated. In addition, nanoparticles can be designed to target specific tissues and cells, which can minimize off-target effects and increase therapeutic efficacy. Therefore, methods that apply various chemical molecule binding methods and antibody-antigen binding for targeting are being developed recently.

Despite the use of various types of nanoparticles for effective protein delivery in the body, limitations still exist. Nanoparticles can be designed to target specific cells or tissues, but they can still accumulate in non-target tissues, thereby causing non-target effects and toxicity. Therefore, in order to solve this problem, methods are being studied to deliver membrane proteins using bio-derived particles such as extracellular vesicles. However, according to Non-Patent Document 3, these biological particles are released outside the cell through a complex biosynthetic pathway compared to synthetic nanoparticles, and thus, they have heterogeneity such as various sizes and differences in protein distribution per individual particle, and have limitations due to low productivity, thereby making it difficult to apply therapeutics.

Additionally, in order to construct bio-derived nanoparticles for targeting, protein expression including target substances is essential, but it is difficult to express proteins with actual target functions due to the complex biosynthetic pathway in cells.

Meanwhile, viruses are largely divided into capsid viruses, which are composed of genetic material such as nucleic acid and a protein shell surrounding the same, and enveloped viruses, which are enveloped once more with a phospholipid bilayer derived from the host. Enveloped viruses have the weakness of lower structural stability compared to capsid viruses, but they contain not only phospholipid membranes derived from the host, but also host-derived proteins, and thus, they have the advantage of higher structural diversity than capsid viruses and better evasion of the host's immune system. Due to these characteristics, enveloped virus infections such as colds and influenza have not disappeared from human society and have persisted for a long time. In addition, it is known that the newly mutated viruses that have recently caused major social problems, such as SARS, MERS, and COVID-19, are also enveloped viruses.

Vaccines are the most effective way to prevent infectious diseases caused by viruses and prevent their spread in society. Vaccines prevent viral infectious diseases by administering virus-related substances into the body such that the human immune system forms a memory for the virus. The development of virus vaccines is carried out by using different methods depending on the type of vaccine, and therefore, the characteristics of the vaccine, such as its immune induction characteristics, time and cost required for development, age at which vaccination is possible, and storage and distribution methods, are also known to differ greatly depending on the type of vaccine.

Traditionally developed and used vaccine technologies are largely divided into inactivated, live-attenuated and subunit vaccines. Virus-like particle (VLP) vaccines using particles with a virus-like shape have also been developed and used, but there have been no successful cases of virus-like particle vaccine development for enveloped viruses. Recently, lipid nano particle (LNP)-based messenger ribonucleic acid (mRNA) vaccine technology and adenovirus-based viral vector vaccine technology have been developed and are widely used worldwide to respond to coronavirus disease-19 (COVID-19). These vaccines are also collectively called nucleic acid vaccines, because they use a method of delivering nucleic acids, which are genetic material containing information on viral antigens, to cells in the body using a carrier such as LNP.

The structure of extracellular vesicles, which are known to be secreted by cells for cell-to-cell communication, is known to be very similar to that of enveloped viruses. Both particles have a membrane structure made of cell-derived lipids, and their sizes are also known to be very similar, at 60-150 nm. In particular, it is known that the biosynthetic processes that cells use to produce certain enveloped viruses and extracellular vesicles are very similar, and based on these studies, it has been speculated that the evolutionary origins of enveloped viruses and extracellular vesicles may be the same. Recently, research is also being conducted to produce enveloped virus-like particles by expressing viral antigens in extracellular vesicles and use the same as vaccines.

Virus-like particles (VLPs) vaccines are developed by expressing viral components to produce and purify nanoparticles that have a structure similar to a virus but lack infectiousness. There have been many successful cases of capsid virus-like particle vaccines, but there have been no successful cases of enveloped virus-like particle vaccines yet. This is because enveloped viruses are produced very heterogeneously through a complex cell-mediated biosynthetic process, unlike capsid viruses that are produced very homogeneously through self-assembly, and it is known that it is very difficult to apply enveloped viruses to the development of virus-like particle vaccines. Due to these characteristics, attempts have been made to develop vaccines in a form that is similar to capsid viruses by binding the antigens of enveloped viruses to self-assembling proteins, but the development of a true enveloped virus-like particle vaccine with a phospholipid bilayer structure has not been achieved to date. In addition, studies on developing enveloped virus-like particles using extracellular vesicles are expected to suffer from the same problems as existing enveloped virus-like particles due to the high heterogeneity of extracellular vesicles.

Meanwhile, as reported in Non-Patent Documents 4 and 5 in the related art, only a few successful cases of VLP vaccines for enveloped viruses such as hepatitis B virus have been developed using membraneless VLPs composed only of viral core proteins such as hepatitis B surface antigens (HBsAg) without a lipid membrane. In particular, NVX-CoV2373, which is a recently approved and commercially available COVID-19 vaccine, is a recombinant nanoparticle-based vaccine lacking a lipid membrane structure, and the protective effect thereof was reported in Non-Patent Document 6.

Under this background, the inventors of the present invention developed a method for preparing nanovesicles which are capable of resolving the homogeneity and productivity problems of plasma membrane bleb-based nanovesicles, and confirmed that, depending on the type of protein expressed in the nanovesicles prepared by the above-described method, the nanovesicles can induce a more enhanced immune response or target specific cells to effectively deliver various antigens or drugs to target cells, thereby completing the present invention.

### RELATED ART DOCUMENTS

### Non-Patent Documents

(Non-Patent Document 1) Missaoui W N, Arnold R D, Cummings B S. Toxicological status of nanoparticles: What we know and what we don't know. Chem Biol Interact. 2018 Nov. 1; 295:1-12.
(Non-Patent Document 2) Elsaesser, Andreas, and C. Vyvyan Howard. “Toxicology of nanoparticles.” Advanced drug delivery reviews 64.2 (2012): 129-137.
(Non-Patent Document 3) Willms E, Cabañas C, Mäger I, Wood M J A, Vader P. Extracellular Vesicle Heterogeneity: Subpopulations, Isolation Techniques, and Diverse Functions in Cancer Progression. Front Immunol. 2018 Apr. 30; 9:738.
(Non-Patent Document 4) McAleer, W. J. et al. Human hepatitis B vaccine from recombinant yeast. Nature 307, 178-180 (1984).
(Non-Patent Document 5) Moradi Vahdat, M. et al. Hepatitis B core-based virus-like particles: A platform for vaccine development in plants. Biotechnology Reports 29, e00605 (2021).
(Non-Patent Document 6) Logue, J., Johnson, R. M., Patel, N. et al. Immunogenicity and protection of a variant nanoparticle vaccine that confers broad neutralization against SARS-CoV-2 variants. Nat Commun 14, 1130 (2023).

### DISCLOSURE

### Technical Problem

Accordingly, an object of the present invention is to provide a method for preparing plasma membrane bleb-based nanovesicles by using a cell line expressing an immunomodulatory protein or a targeting protein.

In addition, another object of the present invention is to provide nanovesicles expressing an immunomodulatory protein or a targeting protein, which are prepared by the above-described method, a pharmaceutical composition and a drug delivery vehicle including the nanovesicles.

Additionally, still another object of the present invention is to provide a method for inducing immunity, including the step of administering the above-described nanovesicles to a subject in need thereof.

Furthermore, still another object of the present invention is to provide a method for signal transduction or targeting in a subject, including the step of administering the above-described nanovesicles to a subject in need thereof.

### Technical Solution

In order to solve the above-described problems, the present invention provides a method for preparing nanovesicles which express an immunomodulatory protein or targeting protein, including the steps of:
(a) establishing a cell line that expresses an immunomodulatory protein or targeting protein inside and outside the plasma membrane;
(b) treating a bleb-inducing agent to a culture medium including the cell line to induce and separate plasma membrane blebs; and
(c) reducing the size of the blebs to generate and separate nanovesicles.

In the present invention, the immunomodulatory protein of step (a) may include at least one selected from the group consisting of (i) to (iv) below:
(i) a pathogenic antigen;
(ii) a signal transduction membrane protein or adaptive immune cell membrane protein marker which is present on the cell membrane and immunochemically involved in adaptive immunity;
(iii) a signal transduction membrane protein or innate immune cell membrane protein marker which is present on the cell membrane and immunochemically involved in innate immunity; and
(iv) a signal transduction membrane protein or membrane protein marker which is expressed on the surface of a tumor.

In the present invention, the pathogenic antigen may be a virus-specific antigen, a bacteria-specific antigen, a parasite-specific antigen or a disease-related human antigen.

In the present invention, the targeting protein may be a membrane protein which is expressed in the tissue or cell to be targeted or a protein that binds thereto.

In the present invention, the cell line of step (a) may be a single antigen-expressing cell line which expresses one virus-specific antigen or a multi-antigen-expressing cell line which expresses one virus-specific antigen and additional other pathogenic antigens or additional other proteins, wherein the additional pathogenic antigen may be a virus-specific antigen, bacteria-specific antigen, parasite-specific antigen, disease-related human antigen or innate immune stimulating antigen which is different from the virus-specific antigen of step (a), and wherein the additional other protein may be at least one selected from the group consisting of a receptor, a ligand and an antibody.

In the present invention, the bleb-inducing agent of step (b) may be treated for 2 to 5 hours.

In the present invention, the bleb-inducing agent of step (b) may be N-ethyl maleimide.

In the present invention, the method for reducing the size of the blebs in step (c) may be at least one method selected from the group consisting of a porous filter extrusion method, an ultrasonic treatment method, a micro-nozzle passage method, a micro-fluid chip passage method and a spray method.

In the present invention, the preparation method may further include the step of (d) purifying nanovesicles.

In the present invention, the step of purifying nanovesicles may be performed by a differential centrifugation method or a density gradient centrifugation method.

In addition, the present invention provides nanovesicles which express an immunomodulatory protein or targeting protein, prepared by the above-described preparation method.

In the present invention, when the immunomodulatory protein is a virus-specific antigen, the nanovesicles may be prepared as plasma membrane bleb-based enveloped virus-mimetic nanovesicles.

In the present invention, the plasma membrane bleb-based enveloped virus-mimetic nanovesicles may express the SARS-CoV-2 Spike protein.

In the present invention, the plasma membrane bleb-based enveloped virus-mimetic nanovesicles may be multivalent virus-mimetic nanovesicles which express multiple antigens.

In the present invention, the immunomodulatory protein may be CD80, and the targeting protein may be a fusion protein including an antibody.

In addition, the present invention provides a vaccine composition, a drug delivery vehicle and a pharmaceutical composition for inducing an immune response, including the above-described nanovesicles.

Additionally, the present invention provides a method for inducing immunity, including the step of administering the above-described nanovesicles to a subject in need thereof.

Furthermore, the present invention provides a method for signaling or targeting in a subject, including the step of administering to the above-described nanovesicles to a subject in need thereof.

### Advantageous Effects

The method for preparing membrane protein-expressing nanovesicles according to the present invention shows an increased nanovesicle yield in terms of the number of particles and the amount of protein compared to existing nanovesicle production methods, and has the advantage of being able to prepare nanovesicles more easily by shortening the time required for preparation. In addition, the nanovesicles prepared by the above method have only plasma membrane components such that the toxicity reduction effect due to intracellular organelles and nucleic acid contamination can be expected, and they have the advantage of being able to uniformly control the size and components compared to other bio-derived nanoparticles. When these nanovesicles are used for protein delivery, they have less toxicity than synthetic nanoparticles, have a longer half-life in the body than when only soluble proteins are injected, and can reduce the number of required injections. In addition, targeting is possible due to the protein on the surface, and additionally, by inducing various forms of protein expression on the outer membrane of cells, the present invention provides an effective protein delivery means as a protein delivery system that can be developed and applied as various protein delivery and therapeutic agents.

The plasma membrane bleb-based enveloped virus-mimetic nanovesicle according to one embodiment of the present invention can be utilized as a new enveloped virus vaccine development platform by solving both the productivity and homogeneity problems of traditional enveloped virus-like particles.

The plasma membrane bleb-based nanovesicle method according to one embodiment of the present invention can be utilized not only for the preparation of virus-mimetic nanovesicles expressing viral antigens, but also for the production of artificial nanovesicles expressing one or more pathogenic microorganisms, disease-specific antigens, mutant antigens, innate immune-inducing antigens and host cell-specific binding molecules, and can be utilized as a versatile vaccine development platform.

Since the enveloped virus-mimetic nanovesicles according to one embodiment of the present invention have a negative surface charge, they can be utilized to produce an effective vaccine composition that electrostatically binds to various cationic adjuvants to enhance immunogenicity while minimizing side effects due to the toxicity of unbound free adjuvants.

The vaccine composition including enveloped virus-mimetic nanovesicles according to one embodiment of the present invention as an active ingredient can be administered by various routes, such as intranasal, subcutaneous, oral or intraperitoneal, in addition to traditional intramuscular injection, and can be utilized to induce various systemic/local immune responses.

The vaccine composition including enveloped virus-mimetic nanovesicles according to one embodiment of the present invention as an active ingredient can very effectively induce the production of systemic/local virus antigen-specific antibodies in experimental animals, and furthermore, it can be utilized as an effective prophylactic vaccine by effectively inducing a cellular immune response that is mediated by antigen-specific T cells.

### DESCRIPTION OF DRAWINGS

FIG. 1 shows the structure of an extruder used to reduce the size of blebs.

FIG. 2 is a schematic diagram showing the induction of T cell activation and inhibitory suppression using CD80, which is a T cell co-stimulatory molecule that induces immune action.

FIG. 3 shows the change in nanovesicle particle concentration and particle size distribution according to the difference in a bleb-inducing agent in the preparation of nanovesicles using a CD80 overexpressing cell line.

FIGS. 4a and 4b show the change in nanovesicle particle concentration and particle size distribution according to the bleb size reduction method in the preparation of nanovesicles using a CD80 overexpressing cell line.

FIG. 5 shows the CD80 protein yield and expression rate according to the difference in a bleb-inducing agent in the preparation of nanovesicles using a CD80 overexpressing cell line.

FIGS. 6a and 6b show the CD80 protein yield and expression rate according to the bleb size reduction method in the preparation of nanovesicles using a CD80 overexpressing cell line.

FIG. 7 shows the change in nanovesicle particle yields according to the differences in a bleb-inducing agent in the preparation of nanovesicles using a CD80 overexpressing cell line.

FIG. 8 shows the change in nanovesicle particle yield according to the bleb size reduction method in the preparation of nanovesicles using a CD80 overexpressing cell line.

FIG. 9 shows the change in CD80 protein expression amount according to the bleb size reduction method in the preparation of nanovesicles using a CD80 overexpressing cell line.

FIGS. 10a to 10f show the results of preparing nanovesicles expressing CD80, and FIGS. 10a and 10b show the results of analyzing CD80 signals and GFP fluorescence signals using a flow cytometry device and immunochemical cell imaging. FIG. 10c shows the results of comparing the amount of CD80 to the amount of the same protein in plasma membrane blebs (Bleb) derived from a CD80 overexpressing cell line and nanovesicles (BNV) using the same. FIG. 10d shows the results of confirming the membrane composition of CD80-expressing nanovesicles through electron microscopy (TEM) imaging. FIG. 10e shows the results of comparing the number of particles with GFP signals compared to the total particles (scatter) of plasma membrane bleb-derived particles (BNV) and conventional extracellular vesicles (EV) using fluorescence nanoparticle tracking analysis. FIG. 10f shows the results of comparing the GFP expression intensity of extracellular vesicles (EV) and plasma membrane bleb-derived particles (BNV) compared to the same amount of protein using Western blot.

FIGS. 11a to Ile show the results of preparing plasma membrane bleb-derived particles (BNV) from cell lines overexpressing fusion proteins including nanobodies for targeting. Specifically, FIG. 11a shows a gene construct for establishing a cell line overexpressing a nanobody fusion protein expressed through a combination of a nanobody for targeting, a membrane protein for positioning the same on the cell membrane, a selected linker and a tag for labeling (tag selection). FIG. 11b shows the results of a Western blot confirming whether the desired fusion protein was expressed in the established cell line. FIG. 11c shows the results of confirming whether the fusion protein expressed in the cell line normally has a cancer-targeting function at the cellular level by treating the cell line expressing the fusion protein with the PSMA antigen, which is a representative marker of prostate cancer, and then treating the same with an antibody targeting the antigen. FIG. 11d shows the results of confirming that the fusion protein was expressed in the nanovesicles (BNV) prepared by inducing plasma membrane blebs from the established cell line and going through a size control and separation process. FIG. 11e shows the results of confirming that BNV exhibits a prostate cancer targeting function by treating PSMA antigen-positive and -negative cell lines with BNV having the fusion protein, respectively, and then treating with an antibody targeting the HA tag contained in the fusion protein, which resulted in more fluorescence signal acquisition in the positive cell line than in the PSMA-negative cell line.

FIG. 12 is a schematic diagram showing the overall process of preparing virus-mimetic nanovesicles (VNV).

FIG. 13a is a representative microscopic image of cell lines, plasma membrane blebs and extruded plasma membrane bleb-derived nanovesicles. The image captured by epifluorescence microscopy shows the cytoplasmic-GFP signal in green and the plasma membrane-mCherry signal in red. The transmission electron microscope image (grayscale) shows Uranyless staining in the dark area.

FIG. 13b shows the bleb yield (N=6), FIG. 13c shows the plasma membrane bleb yield based on cytoplasmic GFP content (N=6), FIG. 13d shows the plasma membrane bleb yield based on plasma membrane mCherry content (N=6), FIG. 13e shows the membrane-to-cytoplasm ratio of blebs (N=6-7), FIG. 13f shows representative microscopic images of plasma membrane blebs isolated at different time points, and FIG. 13g shows the bleb size.

FIG. 14a is a representative transmission electron microscope image of a bleb and a bleb extrudate, FIG. 14b is a schematic diagram showing the size reduction of the bleb extrudate by continuous extrusion, FIGS. 14c and 14d show the size and polydispersity index (n=5) of the extruded plasma membrane blebs measured using dynamic light scattering analysis, FIGS. 14e and 14f show the nanovesicle sizes of bleb extrudates B1000 (FIG. 14e) and B400 (FIG. 14f) measured by nanoparticle tracking analysis (n=3), FIG. 14g shows the size of the plasma membrane blebs extruded through a 200 nm filter (B200) measured using nanoparticle tracking analysis (n=3), and FIG. 14h shows the particle number of the bleb extrudate measured by NTA (n=3).

FIG. 15a shows the membrane-to-cytoplasm ratio of B200 (N>3), and FIG. 15b shows immunoblot results showing the presence of intracellular organelle markers in cells, blebs and B200 lysates.

FIG. 16a shows representative fluorescence microscopy images of cells expressing SARS-CoV-2 spike glycoprotein (S), where cells expressing S WT and S Δ (ERRS) were labeled using antibodies, with green indicating the expression of S.

FIG. 16b shows flow cytometry results of S-GFP stable cell lines, where the gray line indicates wild-type (WT) cells and the red line indicates S-GFP stable cell lines. Bars show the gating used for quantification.

FIG. 16c is a schematic diagram showing the composition of virus-like extracellular vesicles (VEVs) and VNV.

FIG. 16d shows the relative S content per 1 μg of vesicles in terms of GFP signal intensity (n=3).

FIG. 16e shows the results of dot-blot analysis of tetraspanins and S (anti-GFP and anti-RBD) in VEV and VNV lysates.

FIG. 16f shows the results of single-vesicle colocalization analysis of tetraspanins (CD9, CD63, and CD81) and S expression in VEV and VNV, where green represents S-GFP signals, and red represents tetraspanin signals. Image size: 17×17 μm.

FIG. 16g shows the percentage of S-positive vesicles among tetraspanin-positive vesicles (n=9). Bars=mean±SD.

FIG. 17 shows the results of comparing VEV and VNV, where a and b of FIG. 17 show the vesicle yields measured in terms of protein amount (a) and particle number (b), respectively. c of FIG. 17 shows the vesicle purity evaluated by particle number per protein (n=2), and the bars represent the mean±SD. d and e of FIG. 17d show the size of VEV (d) and VNV (e), respectively, evaluated by using NTA (n=3), and the numbers represent the mean±SD, and the solid line represents the average measurement, and the gray area represents the standard deviation.

FIG. 18a is a schematic diagram explaining the VNV binding assay.

FIG. 18b is a representative fluorescence microscopy image of cells treated with VNV, where the green color represents the S-GFP signal of VNV, and the blue color represents the nucleus of the recipient cell. The bars show the gating used for quantification.

FIG. 18c shows the results of quantification of VNV-positive cells by flow cytometry, where the gray line represents WT cells, and the red line represents hACE2-positive cells.

FIG. 19 shows the results of adjuvant evaluation for VNV immunization, where the doses are as follows: VNV 50 μg, alum 250 μg, cholera toxin-beta (CTB) 2.5 μg, cholera toxin (CT) 2.5 μg, wheat germ agglutinin (WGA) 10 μg, and polyethyleneimine (PEI) 10 μg. The bars represent the mean±SD, and the asterisks represent statistically significant deviations compared with the PBS group. * p<0.05, ** p<0.01 (Student's t-test, one-tailed, unequal variances, n=4).

FIGS. 20a to 20e show the results of the VNV vaccine biodistribution study, where FIG. 20a is a schematic diagram of the biodistribution study design, FIG. 20b shows the composition of the intramuscular (IM) and intranasal (IN) vaccines used for the biodistribution study, FIG. 20c shows ex vivo fluorescence images of major organs after IM or IN administration at individual time points, and FIGS. 20d and 20e show the quantification of VNV-related Cy-5.5 signals using the ex vivo fluorescence images. The cropped image area in FIG. 20c was designated as the ROI for signal quantification. Bars=mean±SD. Statistically significant differences compared to the PBS group are indicated by asterisks: * p<0.05, ** p<0.01 (Student's t-test, one-tailed, unequal variances, n=3).

FIG. 21 is a schematic diagram of the VNV vaccination animal study design.

FIGS. 22a to 22f show the results of evaluating the humoral immune response after VNV vaccination, where FIG. 22a is a schematic diagram of the VNV vaccination study design, and FIG. 22b shows the formulation of IM and IN vaccines including various VNV doses utilized in the VNV vaccination study. FIG. 22c shows the evaluation of the body weights of immunized mice, and the body weights were measured immediately before sacrifice and on day 30 after immunization. The bar graphs represent the mean±SD, and the asterisks indicate statistically significant differences compared to the group that did not receive VNV. * p<0.05, ** p<0.01 (Student's t-test, one-tailed, unequal variances; n=4 for IM0 and IN0, n=6 for other doses). FIGS. 22d and 22e show the titers of S-specific serum IgG from mice administered with various IM vaccine doses (d) and mice administered with different IN vaccine doses at various time points (e), respectively. FIGS. 22f and 22g show the titers of S-specific bronchoalveolar lavage fluid (BALF) IgG (f) and BALF IgA (g) of mice administered with IM and IN vaccine doses, respectively, on day 30 after the initial vaccination, bars represent mean±SD, and asterisks represent statistically significant differences compared to the group that did not receive VNV: * p<0.05, ** p<0.01 (Student's t-test, one-tailed, unequal variances; n=4 for IM0 and IN0, n=6 for all other doses). FIG. 22h shows the S-neutralizing titers of serum and BALF extracted from mice administered with the highest VNV dose on day 30, where red dots represent serum results, and blue dots represent BALF results. Bars represent mean±SD. Asterisks indicate statistically significant differences compared to the control group (non-VNV administration): * p<0.05, ** p<0.01 (Student's t-test, one-tailed, unequal variance, n=5). The dotted line indicates the lower detection threshold of the analysis.

FIGS. 23a to 23d show the results of evaluating the cellular immune response to VNV vaccination, where FIGS. 23a and 23b show the quantification of IFN-γ (FIG. 23a) and IL-2 cytokines (FIG. 23b) secreted by splenocytes under S-receptor binding domain (RBD) stimulation, respectively. FIGS. 23c and 23d show representative images (FIG. 23c) and the quantification results (FIG. 23d) of IFN-γ ELISPOT results of splenocytes under S-RBD stimulation, respectively. Cells were isolated from mice administered with IM and IN vaccine doses on day 30 after the initial vaccination. Bars=mean±SD. Statistically significant differences compared to the group that did not receive VNV are indicated by asterisks: * p<0.05, ** p<0.01 (Student's t-test, one-tailed, unequal variances, n=4). The red dashed line indicates the lower detection limit of the assay.

### MODES OF THE INVENTION

Hereinafter, the present invention will be described in more detail.

All technical terms used in the present invention, unless otherwise defined, are used with the same meanings as generally understood by those skilled in the art in the relevant field of the present invention. In addition, although preferred methods or samples are described in the present specification, similar or equivalent ones are also included in the scope of the present invention.

As described above, the current technologies using nanoparticles for protein delivery have limitations such as non-targeting, synthetic materials or toxicity due to overdose. In addition, due to the characteristics of enveloped viruses that are produced very heterogeneously through complex cell-mediated biosynthesis processes, it is known that there has not yet been a case of successfully developing a virus-like particle vaccine for enveloped viruses. Therefore, in the present invention, a solution to the above-mentioned problems was sought by preparing nano-sized vesicles that can be easily recognized in target cells and tissues by using plasma membrane blebs derived from cell lines that overexpress immunomodulatory proteins or targeting proteins on the cell membrane. The method for preparing nanovesicles according to the present invention can provide an increased nanovesicle yield compared to the existing nanovesicle production method by using N-ethyl maleimide (NEM) as a bleb-inducing agent. In addition, by using the prepared nanovesicles, it is possible to develop nanovesicles that have less toxicity than synthetic nanoparticles, have a uniform signal protein delivery compared to existing bio-derived nanovesicles, and have a longer half-life in the body compared to injecting only soluble proteins. By using such protein delivery vehicles, it is possible to expect a reduction in toxicity and side effects due to a reduction in the injection amount and number of injections.

Therefore, the first aspect of the present invention relates to a method for preparing nanovesicles which express an immunomodulatory protein or targeting protein, including the steps of:
(a) establishing a cell line that expresses an immunomodulatory protein or targeting protein inside and outside the plasma membrane;
(b) treating a bleb-inducing agent to a culture medium including the cell line to induce and separate plasma membrane blebs; and
(c) reducing the size of the blebs to generate and separate nanovesicles.

In the present invention, the term “nanovesicles” are prepared by artificially inducing a bleb from the plasma membrane of a cell, and these are distinguished from extracellular vesicles (EVs) that are secreted naturally. For the purpose of the present invention, the nanovesicles according to the present invention may be those in which various immunomodulatory proteins or targeting proteins are expressed. Alternatively, for the purpose of the present invention, the nanovesicles according to the present invention may be those in which a virus-specific antigen and/or additional other antigens or proteins are expressed.

In the present invention, the immunomodulatory protein of step (a) may include at least one selected from the group consisting of (i) to (iv) below:
(i) a pathogenic antigen;
(ii) a signal transduction membrane protein or adaptive immune cell membrane protein marker which is present on the cell membrane and immunochemically involved in adaptive immunity;
(iii) a signal transduction membrane protein or innate immune cell membrane protein marker which is present on the cell membrane and immunochemically involved in innate immunity; and
(iv) a signal transduction membrane protein or membrane protein marker which is expressed on the surface of a tumor.

In the present invention, the pathogenic antigen may be a virus-specific antigen, a bacteria-specific antigen, a parasite-specific antigen or a disease-related human antigen.

In an exemplary embodiment, the cell line of step (a) may be a single antigen-expressing cell line which expresses one virus-specific antigen or a multi-antigen-expressing cell line which expresses one virus-specific antigen and an additional other antigen or an additional other protein.

In an exemplary embodiment, the types of signal-induced membrane proteins or adaptive immune cell membrane protein markers on the cell membrane of cells involved in the immunochemical adaptive immunity may include CD28, CTLA-4, PD-1 (Programmed cell death protein 1), PD-L1 (Programmed death-ligand 1), CD3, CD4, CD8, CD19, CD20, CD40, CD40L (CD40 ligand), B7-1 (CD80), B7-2 (CD86), ICOS (Inducible T-cell co-stimulator), ICOS-L (Inducible T-cell co-stimulator ligand), MHC Class I, MHC Class II, TCR (T-cell receptor), BCR (B-cell receptor), IL-2R (Interleukin-2 receptor), IL-4R (Interleukin-4 receptor), IL-6R (Interleukin-6 receptor), IL-10R (Interleukin-10 receptor), TNF-R (Tumor necrosis factor receptor), IFN-γR (Interferon gamma receptor), TLR4 (Toll-like receptor 4), TLR9 (Toll-like receptor 9), CCR5 (C-C chemokine receptor type 5), CXCR4 (C-X-C chemokine receptor type 4), CD44, CD25 (IL-2 receptor alpha chain), LFA-1 (Lymphocyte function-associated antigen 1), VLA-4 (Very late antigen-4), CD11a/CD18, CD11b/CD18, CD11c/CD18, CD69, CD70, CD134 (OX40), CD137 (4-1BB), CD154 (CD40 ligand), CD161, CD212 (IL-12 receptor), CD223 (LAG3), CD272 (BTLA), CD366 (Tim-3), GITR (Glucocorticoid-induced TNFR-related protein), HLA-DR or HLA-DQ, but the present invention is not limited thereto.

In another exemplary embodiment, the types of signal-induced membrane proteins or innate immune cell membrane protein markers on the cell membrane of cells involved in the innate immunity immunochemically may include TLR1 (Toll-like receptor 1), TLR2 (Toll-like receptor 2), TLR3 (Toll-like receptor 3), TLR5 (Toll-like receptor 5), TLR6 (Toll-like receptor 6), TLR7 (Toll-like receptor 7), TLR8 (Toll-like receptor 8), NOD1 (Nucleotide-binding oligomerization domain-containing protein 1), NOD2 (Nucleotide-binding oligomerization domain-containing protein 2), RIG-I (Retinoic acid-inducible gene I), MDA5 (Melanoma differentiation-associated protein 5), Dectin-1 (C-type lectin domain family 7 member A), Dectin-2, Mannose receptor (CD206), CR3 (Complement receptor 3), CR4 (Complement receptor 4), FcαR (Fc alpha receptor), FcεRI (High-affinity IgE receptor), CD14, CD16 (FcγRIII), CD35 (Complement receptor 1, CR1), CD66b, CD88 (C5a receptor), CLEC-2 (C-type lectin-like receptor 2), NKp30 (Natural killer cell p30-related protein), NKp44 (Natural killer cell protein 44), NKp46 (Natural killer cell protein 46), NKG2D, DNAM-1 (DNAX accessory molecule-1), TREM-1 (Triggering receptor expressed on myeloid cells 1), TREM-2, Siglec-5 (Sialic acid-binding Ig-like lectin 5), Siglec-9, MAC-I (Macrophage-1 antigen), MARCO (Macrophage receptor with collagenous structure), CD180 (RP105, Radioprotective 105), CD205 (DEC-205), CD209 (DC-SIGN), ASGR1 (Asialoglycoprotein receptor 1), ASGR2 (Asialoglycoprotein receptor 2), CD163 (Scavenger receptor cysteine-rich type 1 protein M130), CD303 (BDCA-2, Blood dendritic cell antigen 2), CD324 (E-cadherin), LYVE-1 (Lymphatic vessel endothelial hyaluronan receptor 1), OSCAR (Osteoclast associated receptor), CDw17 (Lactosylceramide), AIM2 (Absent in melanoma 2), FCAR (IgA Fc receptor), CD300, Scavenger receptor A, Scavenger receptor B1, LRP1 (LDL receptor-related protein 1), MD-2, MyD88 (Myeloid differentiation primary response 88) or IRAK4 (Interleukin-1 receptor-associated kinase 4), but the present invention is not limited thereto.

In another exemplary embodiment, the types of signal-induced membrane proteins or membrane protein markers expressed on the surface of the tumor may include EGFR (Epidermal Growth Factor Receptor), HER2/neu (Human Epidermal Growth Factor Receptor 2), VEGFR (Vascular Endothelial Growth Factor Receptor), FGFR (Fibroblast Growth Factor Receptor), PDGFR (Platelet-derived Growth Factor Receptor), MET (Hepatocyte Growth Factor Receptor), CA125 (Cancer Antigen 125), CA19-9 (Cancer Antigen 19-9), CA15-3 (Cancer Antigen 15-3), CEA (Carcinoembryonic Antigen), PSA (Prostate-specific Antigen), MUC1 (Mucin 1), MUC16 (Mucin 16), ALK (Anaplastic Lymphoma Kinase), PSCA (Prostate Stem Cell Antigen), TAG-72 (Tumor-associated Glycoprotein 72), EpCAM (Epithelial Cell Adhesion Molecule), FOLR1 (Folate Receptor 1), CD44v6 (Variant 6 of CD44), CD24, CD30, CD123, CD276 (B7-H3), CTLA-4 (Cytotoxic T-lymphocyte-associated Protein 4), PD-L1 (Programmed Death-Ligand 1), Nectin-4, L1CAM (L1 Cell Adhesion Molecule), ROR1 (Receptor Tyrosine Kinase-like Orphan Receptor 1), TEM1 (Tumor Endothelial Marker 1), TEM8 (Tumor Endothelial Marker 8), IDO1 (Indoleamine 2,3-dioxygenase 1), AXL, MER (c-Mer Proto-oncogene Tyrosine Kinase), TROP-2 (Tumor-associated Calcium Signal Transducer 2), CD70, CD133, CD276 (B7-H3), CD47, S100A9 (Calgranulin B), OX40L (TNF Superfamily Member 4), GITR (Glucocorticoid-induced TNFR-related protein), TIM-3 (T cell Immunoglobulin and Mucin-domain containing-3), LAG-3 (Lymphocyte-activation gene 3), Fas (CD95), FasL (Fas Ligand), DR5 (Death Receptor 5), Galectin-9, CD40, CD40L (CD40 Ligand) or KIT (CD117), but the present invention is not limited thereto.

In an exemplary embodiment, the targeting protein may be a membrane protein expressed in a tissue or cell to be targeted or a protein that binds thereto. Preferably, the targeting protein may include various membrane proteins expressed in tissues and cells present in an in vivo indication to be targeted and proteins having affinity therefor. For example, it includes the membrane expression of antibodies or functional fragments thereof to which antigen-antibody binding is applied, but the present invention is not limited thereto.

In the present invention, the term “functional fragment of antibody” means a fragment having a significant antigen-antibody binding function within an antibody molecule, and includes Fab, F(ab′), F(ab′)₂, Fv, scFv, diabody, triabody or nanobody.

In the present invention, the term “Fab” refers to an antigen-binding antibody fragment, which is a fragment produced by cleaving an antibody molecule with papain, which is a proteolytic enzyme, and it is a dimer of two peptides, VH-CH1 and VL-CL, and the other fragment produced by papain is referred to as Fc (fragment crystalisable).

In the present invention, the term “F(ab′)₂” refers to a fragment including an antigen binding site among fragments generated by cleaving an antibody with pepsin, which is a proteolytic enzyme, and it represents a tetramer in which two Fabs are linked by a disulfide bond. The other fragment generated by pepsin is referred to as pFc′.

In the present invention, the term “Fab” refers to an antibody fragment having a structure similar to the Fab, which is generated by reducing the F(ab′)₂, and has a slightly longer length of the heavy chain portion compared to the Fab.

In the present invention, the term “Fv (fragment variable)” refers to a dimeric antibody fragment composed only of the variable regions (VH and VL) of the heavy and light chains of the antibody, which is intermediate in size between the nanobody and the Fab (approximately ˜25 kD), and it is produced by proteolytic hydrolysis of the antibody under special conditions or by inserting a gene encoding the VH and VL into one expression vector and expressing the same.

In the present invention, the term “scFv” refers to a single chain fragment variable, and it means a recombinant antibody fragment which is prepared as a single chain using a linker for the variable regions (VH and VL) of the Fab of an antibody.

In the present invention, the term “diabody” refers to a recombinant antibody fragment having divalent bispecificity prepared by shortening the linker length between the VH and VL of scFv (5 a.a) such that two scFvs form a dimer with each other, and it is known to have higher antigen specificity than conventional scFvs.

In the present invention, the term “triabody” refers to a trivalent recombinant antibody fragment prepared such that three scFvs form a trimer with each other.

In the present specification, the term “nanobody” refers to a single domain antibody analogue which is composed of only one heavy chain variable region, and has the same meaning as “single domain antibody (VHH antibody)” or “synthetic nanoantibody (sybody)” and can be used interchangeably. Nanobody is the smallest antigen-binding fragment with full functionality, and is usually constructed by first obtaining an antibody naturally lacking light chain and heavy chain constant region 1 (CH1), and then cloning the variable region of the antibody heavy chain to construct a single domain antibody (Variable Domain of Heavy-chain Antibodies, VHH) which is composed of only one heavy chain variable region. Nanobody can be prepared by artificially processing antibody fragments extracted from camelids such as camels, llamas and alpacas, and has the advantage of being strong against external environments such as temperature changes, easy to administer and having a high production yield because it has a size of one-tenth of the size of a general antibody.

In the present invention, the type of cell line in step (a) can be any organism, preferably, a mammal, for example, a human cell. The cell line is transformed or transfected to produce an exogenous protein. In an exemplary embodiment, the exogenous protein may be an immunomodulatory protein and/or a targeting protein as described above. In another exemplary embodiment, the exogenous protein may be one or more virus-specific antigens and/or one or more additional other proteins.

In the present invention, the “transformation” or “transfection” means a modification of the cell genotype by introduction of an exogenous polynucleotide, and means that the exogenous polynucleotide is introduced into the cell regardless of the method used for the transformation. The exogenous polynucleotide may be inserted into a vector and used for transformation or transfection of the cell line. In the present invention, the “vector” is used for the purpose of replication or expression of the exogenous polynucleotide, and generally includes one or more of a signal sequence, an origin of replication, one or more marker genes, enhancers, promoters and a transcription termination sequence. A plasmid, which is a type of vector, is a linear or circular double-stranded DNA molecule into which foreign polynucleotide fragments can be linked.

In the present invention, the vector may be understood to have the same meaning as an “expression vector”, which is a type of vector that is capable of expressing the polynucleotide. One polynucleotide sequence is “operably linked” to a regulatory sequence if the regulatory sequence affects the expression (e.g., level, timing or location of expression) of the polynucleotide sequence. The regulatory sequence is a sequence that affects the expression (e.g., level, timing or location of expression) of a nucleic acid to which it is operably linked. The regulatory sequence may, for example, exert its influence directly on the regulated nucleic acid or through the action of one or more other molecules (e.g., polypeptides that bind to the regulatory sequence and/or the nucleic acid). The regulatory sequence includes a promoter, an enhancer and other expression control elements.

The vector can be introduced into the cell by an exogenous polynucleotide by a method known in the art, such as, but not limited to, transient transfection, microinjection, transduction, fusion, calcium phosphate precipitation, liposome-mediated transfection, DEAE dextran-mediated transfection, polybrene-mediated transfection, electroporation, a gene gun or a known method for introducing nucleic acids into cells, thereby transforming the cell. In the present invention, the “expression” means that a protein or a polypeptide is produced in the cell.

In the present invention, the “exogenous protein” means a protein or peptide that is not endogenously expressed in the cell, and it refers to a protein or peptide that is expressed inside and outside the plasma membrane of the cell by artificial transformation or transfection. The exogenous protein can be expressed in a cell by transforming or transfecting the cell with a vector into which a polynucleotide encoding the above protein has been inserted.

In the present invention, the additional other pathogenic antigen may be a virus-specific antigen or an innate immune stimulating antigen other than the virus-specific antigen of step (a).

For example, the innate immune stimulating antigen may be a damage-associated molecular pattern (DAMP), a pathogen-associated molecular pattern (PAMP) or a combination thereof, but the present invention is not limited thereto.

In the present invention, the additional other protein may be at least one selected from the group consisting of a receptor, a ligand and an antibody, but the present invention is not limited thereto.

In the present invention, the cell line of step (a) may express other pathogenic antigens in addition to one virus-specific antigen. The pathogenic antigen may include bacteria-specific antigens, parasite-specific antigens or cancer-specific antigens, but the present invention is not limited thereto.

In an exemplary embodiment, the multi-antigen expressing cell line may express two or more selected from the group consisting of virus-specific antigens, bacteria-specific antigens, parasite-specific antigens and cancer-specific antigens.

In another exemplary embodiment, the multi-antigen expressing cell line may simultaneously express the pathogenic antigen and an innate immune stimulating antigen that acts as an immune enhancer.

In another exemplary embodiment, the multi-antigen expressing cell line may simultaneously express any one or more selected from the group consisting of a receptor, a ligand and an antibody that specifically binds to the pathogenic antigen and a host cell.

In this way, since the cell line of step (a) can simultaneously express two or more virus-specific antigens on the plasma membrane, the method of the present invention can also be applied as a method for producing multivalent virus-mimetic nanovesicles.

In addition, since the cell line of step (a) can simultaneously express pathogenic antigens and innate immune stimulating antigens on the plasma membrane, the method of the present invention can also be applied as a method for preparing immune-enhancing virus-mimetic nanovesicles.

In addition, since the cell line of the step (a) can simultaneously express at least one selected from the group consisting of a pathogenic antigen and a receptor, a ligand and an antibody, the method of the present invention can also be applied as a method for preparing target virus-mimetic nanovesicles.

In the present invention, the target virus-mimetic nanovesicles can target a specific cell through a receptor, a ligand or an antibody expressed on the surface of the nanovesicles, and can effectively deliver a virus-specific antigen to the target cell to induce an immune response in the body. Examples of the target cell include antigen presenting cells (APCs) such as B cells, macrophages and dendritic cells, but the present invention is not limited thereto.

In the present invention, the bleb-inducing agent of the step (b) can be treated for 2 to 5 hours, and preferably, 2 to 4 hours. If the bleb-inducing agent is treated for less than 2 hours, not only will plasma membrane blebs not be sufficiently induced, but also the content of target proteins (i.e., virus-specific antigens and/or additional other proteins) within the blebs may be significantly reduced. In addition, if the bleb-inducing agent is treated for more than 5 hours, the adverse effects of protein denaturation and expression of apoptosis-related molecules are greater than the benefit of improving the bleb yield according to the increase in time, and thus, the effect of improving the bleb yield according to the time is minimal, and the size of the formed blebs may even increase.

In the present invention, the bleb-inducing agent of step (b) may include at least any one selected from the group consisting of a sulfhydryl blocking agent, such as formaldehyde, pyruvate, acetaldehyde, glyoxal, glutaraldehyde, acrolein, methacrolein, pyridoxal, N-ethyl maleimide (NEM), maleimide, chloromercurybenzoate, iodoacetate, potassium absinthe, sodium selenite, thimerosal (merthiolate), benzoyl peroxide, cadmium chloride, hydrogen peroxide, iodobenzoic acid, sodium meraluride (mercuryhydrin), mercury (II) chloride, mercuric chloride, chlormerrodrin (neohydrin), phenylhydrazine, potassium tellurate, sodium malonate, p-arsenobenzoic acid, 5,5′-diamino-2,2′-dimethyl arsenobenzene, N,N′-dimethylene sulfonate disodium salt, iodoacetamide, oxophenarcin (maphacene), gold chloride, p-chloromercurybenzoic acid, p-chloromercuryphenylsulfonic acid, cupric chloride, iodine, merbromine (mercurochrome), porphyrindine, potassium permanganate, mersaleil (salirrhagne), silver nitrate, strong silver protein (protargol), uranyl acetate and dithiothreitol (DTT). Preferably, the bleb-inducing agent of step (b) may be N-ethyl maleimide.

The bleb-inducing agent may be dissolved in a buffer and treated with a cell medium. Therefore, the bleb-inducing agent treatment time may mean the time for culturing cells in a medium including the bleb-inducing agent.

In an exemplary embodiment, when N-ethyl maleimide is used as the bleb-inducing agent in step (b), N-ethyl maleimide may be treated at an appropriate concentration range depending on the cell type. For example, when N-ethyl maleimide is treated to HEK293 and HEK293T cell lines, the concentration may be 0.5 to 5 mM, and preferably, 1 to 3 mM.

In the present invention, when preparing nanovesicles using N-ethyl maleimide as a bleb-inducing agent in the step (b), the amount of bleb produced increases compared to using DTT and PFA such that the amount of target protein itself increases, and nanovesicles expressing the target protein can be obtained with a higher recovery rate. For example, when preparing nanovesicles using N-ethyl maleimide as a bleb-inducing agent, the recovery rate of target protein that is at least 2 times higher, 3 times higher, 4 times higher, or 3 to 4.5 times higher can be achieved compared to using DTT and PFA. In addition, when preparing nanovesicles using N-ethyl maleimide as a bleb-inducing agent, the nanovesicle particle yield that is at least 1.5 times higher, 1.7 times higher, or 1.5 to 2 times higher can be achieved compared to using DTT and PFA.

In the present invention, in addition to treating a bleb-inducing agent, the step (b) may induce and separate blebs by using a hypotonic buffer and a hypertonic buffer.

In the present invention, the plasma membrane blebs obtained after step (b) may be reduced in size through various methods to generate nanovesicles. The method for reducing the size of the blebs in step (c) may be at least one method selected from the group consisting of a porous filter extrusion method, a sonication method, a micro-nozzle passage method, a microfluidic chip passage method and a spray method, but the present invention is not limited thereto. Preferably, the porous filter extrusion method may be performed one or more times to adjust the size (diameter) of the plasma membrane blebs to a size of 200 nm or less. For example, the prepared plasma membrane blebs may be reduced in size by continuously extruding the same through a porous filter whose pore diameters are sequentially reduced. In this case, the pore diameter of the porous filter used for the first extrusion may be 800 to 1,200 nm, the pore diameter of the porous filter used for the second extrusion may be 400 to 800 nm, and the pore diameter of the porous filter used for the third extrusion may be 100 to 400 nm, but the present invention is not limited thereto. The preferred number of extrusions may be 5 to 12 times for each filter. For example, extrusion may be performed 5 to 12 times in a porous filter having a pore diameter of 800 to 1,200 nm, then extrusion may be performed 5 to 12 times in a porous filter having a pore diameter of 400 to 800 nm, and finally extrusion may be performed 5 to 12 times in a porous filter having a pore diameter of 100 to 400 nm.

In the present invention, the method for preparing the nanovesicle may further include a purification step to maximize the content of the immunomodulatory protein or targeting protein expressed in the nanovesicles while minimizing the content of contaminants such as cytoplasm and cell organelles, if necessary. The purification step may be performed by a differential centrifugation method or a density gradient centrifugation method, but may be performed without limitation by any method known to be capable of purifying the nanovesicles.

The method for preparing the nanovesicles according to the method of the present invention can produce nanovesicles with significantly improved homogeneity compared to existing enveloped virus-mimetic particle production methods with high productivity.

In addition, the present invention can provide a method for preparing multifunctional virus-mimetic nanovesicles by grafting two or more of the aforementioned virus-mimetic nanovesicle preparation methods.

In another aspect, it is possible to provide a method for preparing plasma membrane artificial nanovesicles by using a cell line expressing a pathogenic microorganism-derived antigen or a cell line expressing a disease-related human antigen instead of a cell line expressing a virus-specific antigen in step (a).

The second aspect of the present invention relates to nanovesicles expressing an immunomodulatory protein or a targeting protein prepared by the above-described preparation method.

In the present invention, when the immunomodulatory protein is a virus-specific antigen, the nanovesicles may be prepared as plasma membrane bleb-based enveloped virus-mimetic nanovesicles.

The plasma membrane-based enveloped virus-mimetic nanovesicles prepared by the above-described method may be plasma membrane artificial nanovesicles prepared by using a cell line expressing a virus-specific antigen on the plasma membrane as a material.

In an exemplary embodiment, when the intracellular expression site of the virus-specific antigen expressed on the surface of the plasma membrane-based enveloped virus-mimetic nanovesicles is the plasma membrane, the antigen may be used as is without additional sequence modification.

In another exemplary embodiment, when the intracellular expression site of the virus-specific antigen expressed on the surface of the plasma membrane-based enveloped virus-mimetic nanovesicles is not the plasma membrane, an appropriate genetic modification is required such that the antigen can be expressed on the plasma membrane.

In a specific embodiment, the plasma membrane-based enveloped virus-mimetic nanovesicles may be those in which the SARS-CoV-2 Spike protein is expressed. In this case, since the SARS-CoV-2 Spike protein must be expressed on the plasma membrane of the cell, it is preferable that the endoplasmic reticulum retention signal (ERRS) located at the C-terminus of the wild-type (WT) Spike protein is lacking.

The sequence information of the SARS-CoV-2 Spike protein can be found in an open database such as NCBI. For example, the amino acid sequence of the SARS-CoV-2 Spike protein may be an amino acid sequence disclosed in GenBank: QIG55857.1, GenBank: QUJ10661.1, GenBank: QUJ10660.1 and the like, or a part thereof. In addition, the nucleotide sequence encoding the SARS-CoV-2 spike protein may be a nucleotide sequence disclosed in Gene ID: 43740568 and the like or a part thereof.

In a specific embodiment, the amino acid sequence of the SARS-CoV-2 Spike protein used in the present invention may be, for example, a sequence in which 19 amino acid residues are deleted from the C-terminus of the amino acid sequence disclosed in GenBank: QIG55857.1.

In the present invention, the plasma membrane bleb-based enveloped virus-mimetic nanovesicles may be multivalent virus-mimetic nanovesicles expressing multiple antigens.

In an exemplary embodiment, the plasma membrane bleb-based enveloped virus-mimetic nanovesicles may be multivalent virus-mimetic nanovesicles prepared by using a multi-antigen expressing cell line that simultaneously expresses two or more virus-specific antigens on the plasma membrane.

In another exemplary embodiment, the plasma membrane bleb-based enveloped virus-mimetic nanovesicles may be immune-enhancing virus-mimetic nanovesicles prepared by using a multi-antigen expressing cell line that simultaneously expresses a pathogenic antigen and an innate immune-stimulating antigen on the plasma membrane.

In still another exemplary embodiment, the plasma membrane bleb-based enveloped virus-mimetic nanovesicles may be target virus-mimetic nanovesicles prepared by using a multi-antigen expressing cell line that simultaneously expresses a pathogenic antigen and at least one selected from the group consisting of a receptor, a ligand and an antibody.

In still another exemplary embodiment, the plasma membrane bleb-based enveloped virus-mimetic nanovesicles may be multifunctional virus-mimetic nanovesicles prepared by grafting two or more of the aforementioned preparation methods.

In another aspect, the present invention may provide plasma membrane-based artificial nanovesicles prepared by using a cell line expressing a pathogenic microbial antigen or a cell line expressing a disease-related human antigen instead of a cell line expressing a virus-specific antigen.

In an exemplary embodiment, the surface of the plasma membrane artificial nanovesicles expresses a pathogenic microorganism-derived antigen or a disease-related human antigen. Herein, when the intracellular expression site of the pathogenic microorganism-derived antigen or the disease-related human antigen is the plasma membrane, the corresponding antigen may be used as is without additional sequence modification.

In another exemplary embodiment, when the intracellular expression site of the pathogenic microorganism-derived antigen or the disease-related human antigen is not the plasma membrane, appropriate genetic modification is required such that the antigen can be expressed on the plasma membrane.

In the present invention, additional other proteins such as receptors, ligands or antibodies that are capable of targeting specific cells are expressed on the surface of the plasma membrane artificial nanovesicles such that various antigens can be delivered to target cells more effectively.

The nanovesicles according to the present invention may be a new type of enveloped virus-mimetic nanovesicles in which the antigen source is very homogeneous and has a size and structure that are similar to an enveloped virus but has no infection ability. Such enveloped virus-mimetic nanovesicles have the same or similar size and structure as an enveloped virus and exhibit significantly improved homogeneity compared to existing enveloped virus-mimetic particle technologies.

The virus-mimetic nanovesicles (VNV) derived from the cell-engineered plasma membrane according to the present invention exhibit a vesicle structure similar to that of a native enveloped virus, and have been demonstrated to have the ability to efficiently induce immune responses. VNV is generated by extruding plasma membrane blebs supplied from an in vitro cell line stably expressing a viral antigen.

In the present invention, through characterization, it was confirmed that VNV has structural characteristics similar to that of an enveloped virus, and is highly enriched in plasma membrane material without material from other cell organelles. In addition, VNV exhibited superior viral antigen integration and expression homogeneity compared to EV naturally secreted from the same cell line. In order to evaluate the vaccine potential of VNV, severe acute respiratory syndrome coronavirus 2 (SARS-CoV-2) VNV and VNV vaccine solution prepared with an adjuvant were administered to mice via intramuscular or intranasal routes. The ability to induce humoral and cellular responses to SARS-CoV-2 S glycoprotein (S) was evaluated. This vaccination successfully induced the production of S-specific IgG and IgA antibodies that neutralize the binding of S protein to the human angiotensin converting enzyme 2 (hACE2) receptor. In addition, splenocytes isolated from vaccinated mice showed S-specific interferon-γ and IL-2 cytokine responses, indicating the successful induction of cellular immune responses by VNV vaccination. Preclinical evaluations in mice successfully demonstrated the clinical potential of VNV as a prophylactic vaccine against enveloped virus infections.

The nanovesicles prepared by the above-described method may be plasma membrane artificial nanovesicles prepared by using a cell line expressing an immunomodulatory protein and/or targeting protein on the plasma membrane as a material.

In the present invention, the immunomodulatory protein may be a signal transduction protein.

The signal transduction protein includes signal-inducing membrane proteins on the cell membranes of cells involved in adaptive immunity immunochemically, adaptive immune cell membrane protein markers and the like, but the present invention is not limited thereto. Since the specific types of signal-induced membrane proteins or adaptive immune cell membrane protein markers in the cell membrane of cells involved in the above immunochemical adaptive immunity are the same as described above, the descriptions thereof will be omitted.

In the present invention, the signal transduction protein includes signal-induced membrane proteins in the cell membrane of cells involved in the above immunochemical innate immunity, innate immune cell membrane protein markers and the like, but the present invention is not limited thereto. Since the specific types of signal-induced membrane proteins or innate immune cell membrane protein markers in the cell membrane of cells involved in the above immunochemical innate immunity are the same as described above, the descriptions thereof will be omitted.

In the present invention, the signal transduction protein includes signal-induced membrane proteins, membrane protein markers and the like that are expressed on the surface of a tumor, but the present invention is not limited thereto. Since the specific types of signal-induced membrane proteins or membrane protein markers that are expressed on the surface of a tumor are the same as described above, the descriptions thereof will be omitted.

In the present invention, the cells involved in the adaptive immunity or innate immunity include antigen-presenting cells (APCs) such as B cells, macrophages and dendritic cells, T cells, NK cells, mast cells, neutrophils, eosinophils or basophils, but the present invention is not limited thereto.

In an exemplary embodiment, the nanovesicles may be nanovesicles expressing CD80.

The CD80 may be found on the surface of various immune cells including B cells, monocytes or T cells, but is most commonly found on antigen-presenting cells (APCs) such as dendritic cells, and plays an important role in regulating T cell immune function as a checkpoint protein of the immunological synapse. CD80 is a ligand for the proteins CD28 and CTLA-4 found on the surface of T cells. The interaction of CD80 and CD28 triggers a co-stimulatory signal and enhances and sustains T cell activation. In contrast, the antagonistic interaction of CD80 and CTLA-4 inhibits some of the T cell effector functions.

In a specific exemplary embodiment of the present invention, nanovesicles that induce T cell activation and suppression inhibitory action were constructed by using CD80, which is a T cell co-stimulatory molecule that induces immune responses. To this end, plasma membrane blebs were generated by using a cell line overexpressing CD80, and the shape and homogeneity of nanovesicles that underwent size control and separation processes were determined.

The sequence information of the CD80 protein can be found in open databases such as NCBI. For example, the amino acid sequence of the human CD80 protein may be an amino acid sequence disclosed in UniProtKB/Swiss-Prot: P33681.1, NCBI Reference Sequence: NP_005182.1, GenBank: AAH42665.1 and the like, or a part thereof. In addition, the nucleotide sequence encoding the CD80 protein may be a nucleotide sequence disclosed in NCBI Reference Sequence: NM_005191.4, GenBank: EF064750.1, GenBank: BC042665.1 and the like, or a part thereof.

In a specific embodiment, the amino acid sequence of the CD80 protein used in the present invention may be an amino acid sequence encoded by, for example, the nucleotide sequence of SEQ ID NO: 1 or 2.

In the present invention, the nanovesicles may be nanovesicles expressing a fusion protein including an antibody for targeting or a functional fragment thereof. Since the description of the functional fragment of the antibody is the same as described above, the description thereof will be omitted.

In the present invention, the antibody for targeting or a functional fragment thereof may be combined with an appropriate membrane protein such that it can be expressed on the plasma membrane of a cell. For example, the membrane protein that enables the antibody or a functional fragment thereof for targeting to be expressed on the plasma membrane of a cell may be Integrin alpha1 (ITGA1), Lysosome-associated membrane protein 2B (LAMP2B), CD4, Insulin Receptor, TNF Receptor (TNFR) and the like, but the present invention is not limited thereto.

In still another specific exemplary embodiment of the present invention, nanovesicles targeting prostate cancer were constructed by generating plasma membrane bleb-derived nanovesicles from a cell line overexpressing a fusion protein including a nanobody targeting prostate-specific membrane antigen (PSMA). In this case, the nanobody may be combined with an appropriate membrane protein such that it can be expressed on the plasma membrane. In a specific exemplary embodiment of the present invention, the membrane protein region of the Integrin Alpha1 (ITGA1) protein was used as the membrane protein for expressing the nanobody on the plasma membrane. The nanobody and ITGA1 can be connected with a peptide linker.

In the present invention, the peptide linker refers to a commonly used GS (Gly-Ser) linker. For example, it may be (GS)n, (GGS)n, (GGGS)n or (GGGGS)n, and preferably (GGGGS)n. In this case, n can be an integer from 1 to 5, but the present invention is not limited thereto.

In a specific embodiment, the amino acid sequence of the fusion protein including the nanobody used in the present invention may be, for example, an amino acid sequence encoded by the nucleotide sequence of SEQ ID NO: 3.

Through the above two examples, it was confirmed that homogeneous membrane protein expression cell line-derived vesicles (BNVs) can be constructed, and signal transduction and targeting can be performed by using the same.

The nanovesicles produced by the method of the present invention are not particularly limited as long as their diameter is in the nanometer range, but may have a size (diameter) of 100 to 300 nm, and preferably, 100 to 200 nm.

The nanovesicles prepared by the method of the present invention are composed of plasma membrane components and cytoplasm, and do not include other cell organelles. More preferably, the plasma membrane components are characterized by showing a higher content than the cytoplasm.

The membrane protein-expressing nanovesicles prepared by the preparation method of the present invention may induce an immune response of immune cells by themselves, or may show therapeutic activity by additionally expressing a therapeutic protein in the nanovesicles, or by including an additional drug in the nanovesicles.

Therefore, the third aspect of the present invention relates to a vaccine composition, a drug delivery vehicle or a pharmaceutical composition for inducing an immune response, including the above-described nanovesicles.

In one embodiment, the present invention relates to a vaccine composition, including the above-described plasma membrane bleb-based enveloped virus-mimetic nanovesicles.

In an exemplary embodiment, the vaccine composition of the present invention may include plasma membrane bleb-based enveloped virus-mimetic nanovesicles expressing the SARS-CoV-2 spike protein. The plasma membrane bleb-based enveloped virus-mimetic nanovesicles containing the SARS-CoV-2 spike protein may induce systemic and/or local immune responses depending on the route of administration, may induce the production of antibodies that are specific to the SARS-CoV-2 spike protein, and may also induce a cellular immune response mediated by T cells that is specific to the SARS-CoV-2 spike protein. Therefore, the vaccine composition may effectively prevent SARS-CoV-2 infection.

In the present invention, the vaccine composition may further include an immune enhancer. The immune enhancer (i.e., vaccine adjuvant or adjuvant) refers to a pharmaceutical or immunological agent which is administered for the purpose of enhancing the immune response of the vaccine, that is, increasing the immune response in the body by the antigen.

In the present invention, the immune enhancer may be a non-self substance showing a unique affinity for the plasma membrane. For example, the immune enhancer may include any one or more selected from the group consisting of pathogenic nucleic acids, cationic lipids, bacteria-derived toxins, plant-derived agglutinins and attenuated derivatives thereof, as a non-self substance showing a unique affinity for the plasma membrane, but the present invention is not limited thereto. For example, aluminum hydroxide, aluminum phosphate, Alum, MF59, virosome, AS04 [a mixture of aluminum hydroxide and monophosphoryl lipid A (MPL)], AS0₃ (a mixture of DL-α-tocopherol, squalene and polysorbate 80), CpG DNA, flagellin, Poly I:C, AS01 (a mixture of QS-21, MPL and liposomes), AS0₂ [a mixture of QS-21, MPL and oil-in-water emulsion], immune stimulating complexes (ISCOMs) and ISCOM matrix may be used, but the present invention is not limited thereto. These immune enhancers may be present in a form that is bound to the plasma membrane bleb-based enveloped virus-mimetic nanovesicles by electrostatic forces, or may remain in the solution without being bound.

The vaccine composition according to the present invention may be formulated in a suitable form with a pharmaceutically acceptable carrier. Pharmaceutically acceptable carriers include, for example, carriers for parenteral administration such as water, suitable oils, saline, aqueous glucose and glycols, and may further include stabilizers and preservatives. Suitable stabilizers include antioxidants such as sodium bisulfite, sodium sulfite or ascorbic acid. Suitable preservatives include benzalkonium chloride, methyl- or propyl-paraben and chlorobutanol. In addition, the vaccine composition according to the present invention may appropriately include suspending agents, solubilizing agents, stabilizers, isotonic agents, preservatives, antiadsorbents, surfactants, diluents, excipients, pH adjusters, analgesics, buffers, antioxidants and the like, depending on the administration method or formulation thereof. Pharmaceutically acceptable carriers and formulations that are suitable for the present invention, including those exemplified above, are described in detail in the literature [Remington's Pharmaceutical Sciences, latest edition]. The dosage of the vaccine composition to a patient varies depending on many factors including the patient's height, body surface area, age, active ingredient administered, gender, time and route of administration, general health and other drugs administered simultaneously.

The vaccine composition of the present invention may be administered in a pharmaceutically effective amount. As used herein, the term “pharmaceutically effective amount” means an amount that is sufficient to treat a disease at a reasonable benefit/risk ratio applicable to medical treatment, and the effective dosage level may be determined based on factors including the type and severity of the individual, age, gender, activity of the drug, sensitivity to the drug, time of administration, route of administration and excretion rate, duration of treatment, concurrently used drugs and other factors well known in the medical field. The vaccine composition of the present invention may be administered in a dosage of 0.1 mg/kg to 1 g/kg, and is preferably administered in a dosage of 1 mg/kg to 500 mg/kg. Meanwhile, the dosage may be appropriately adjusted according to the patient's age, gender and condition.

The vaccine composition may be administered to a subject through various routes to induce the expression of antigen-specific antibodies to the antigen. The route of administration of the vaccine composition may be administered through any general route as long as it can reach the target tissue. For example, it may be administered through the nasal, oral, subcutaneous, intramuscular, intraperitoneal and intraperitoneal routes, but the present invention is not limited thereto. The vaccine composition of the present invention may be administered through the above-mentioned route at least once.

The term “subject” in the present invention includes a human or a non-human animal. The term “non-human animal” may be a vertebrate, such as a non-human primate, a monkey, a sheep, a cow, a horse, a pig, a cat, a dog, a rabbit, a camel and a rodent (e.g., a mouse, a rat and a guinea pig). The subject may preferably be a human. The term “subject” may be used interchangeably with “individual” and “patient” in the present specification.

In an exemplary embodiment, the vaccine composition of the present invention may include multivalent virus-mimetic nanovesicles expressing the multiple antigens. The above multiple antigens may be two or more pathogenic antigens.

In another exemplary embodiment, the vaccine composition of the present invention may include the target virus-mimetic nanovesicles.

In another embodiment, the present invention relates to a pharmaceutical composition or drug delivery vehicle for inducing an immune response, including the above-described nanovesicles.

The term “drug delivery vehicle” as used in the present invention means any means or act of expressing and delivering an additional therapeutic protein in the nanovesicles according to the present invention to deliver a drug to a specific organ, tissue, cell or cell organelle, or loading the drug and delivering the same.

The pharmaceutical composition according to the present invention may include a pharmaceutically acceptable carrier. Since the description of the pharmaceutically acceptable carrier is the same as described above, the description thereof will be omitted.

The pharmaceutical composition of the present invention may be be administered orally or parenterally (e.g., intravenously, subcutaneously or applied to the skin) depending on the intended method, and the dosage varies depending on the patient's condition and weight, the degree of the disease, the drug form, the administration route and the time, but it may be appropriately selected by those skilled in the art.

The pharmaceutical composition according to the present invention is administered in a pharmaceutically effective amount. Since the description of the pharmaceutically effective amount is the same as described above, the description thereof will be omitted.

The composition according to the present invention may be applied to prevent, improve or treat various diseases depending on the type of antigen expressed in the nanovesicles.

Therefore, the fourth aspect of the present invention relates to a method of vaccination, including administering an effective amount of the above-described vaccine composition to a subject in need thereof.

In the present invention, the term “effective amount” refers to an amount that exhibits a greater response than a negative control group, and preferably refers to an amount that is sufficient to prevent, improve or treat a specific disease.

In the present invention, since the description of the effect, administration route, number of administrations and administration amount of the vaccine composition administered to the subject is the same as described above, the descriptions thereof will be omitted.

In the present invention, the vaccine composition may be administered in combination with an additional drug for the purpose of increasing the efficacy of the vaccine or reducing side effects.

In an exemplary embodiment, the vaccine composition administered to the subject may include plasma membrane bleb-based enveloped virus-mimetic nanovesicles expressing the SARS-CoV-2 spike protein. The plasma membrane bleb-based enveloped virus-mimetic nanovesicles expressing the SARS-CoV-2 spike protein may induce systemic and/or local immune responses depending on the route of administration, may induce the production of antibodies that are specific to the SARS-CoV-2 spike protein, and may also induce a cellular immune response mediated by T cells specific to the SARS-CoV-2 spike protein. Therefore, the vaccination method of the present invention may include a method for preventing SARS-CoV-2 infection.

The vaccination method according to the present invention may be applied to prevent, improve or treat various diseases depending on the type of antigen expressed in the nanovesicles included in the vaccine composition. Therefore, the vaccination method of the present invention may include a method of preventing a related disease through an immune response induced by a specific antigen expressed in nanovesicles, a specific antibody produced by the specific antigen, and a cellular immune response mediated by the specific antigen-specific T cell.

The composition according to the present invention may be utilized as a method of inducing an immune response, and a method of applying a signaling effect or targeting effect of nanovesicles, depending on the type of signaling protein and/or targeting protein expressed in the nanovesicles.

Therefore, the fifth aspect of the present invention relates to a method of inducing immunity and a method of signaling or targeting in the subject, including the step of administering the above-described nanovesicles to a subject in need thereof.

In the present invention, the method of applying the signal transduction effect or targeting effect includes a method of measuring the binding force of a membrane protein (e.g., a receptor) expressed in nanovesicles with a corresponding substance (e.g., a ligand), but the present invention is not limited thereto.

In the present invention, the method of measuring the binding force may include an in vitro experiment or in vivo measurement of the binding force between a cell having a membrane protein expressed in nanovesicles and a corresponding substance.

In the present invention, the method of applying the signal transduction effect or targeting effect includes measuring changes in the genetic material, protein expression and the like of cells due to downstream signal transduction between a membrane protein expressed in nanovesicles and a cell having a corresponding substance in vitro or in vivo, but the present invention is not limited thereto.

In the present invention, the method of applying the signal transduction effect or targeting effect includes measuring the targeting effect and in vivo duration due to a membrane protein expressed in a vesicle membrane when administering a nanovesicle in vivo, but the present invention is not limited thereto.

In the present invention, the method of applying the signaling effect or targeting effect includes measuring protein delivery efficiency (targeting) according to the administration route when administering the nanovesicle in vivo, but the present invention is not limited thereto.

In the present invention, the method of applying the signaling effect or targeting effect includes measuring protein delivery efficiency and toxicity effect according to the administration dose or number of administrations when administering the nanovesicle in vivo, but the present invention is not limited thereto.

Hereinafter, the present invention will be described in more detail through examples. However, the present invention can be modified in various ways and can have various forms, and the specific examples and descriptions described below are only intended to help understanding the present invention, and are not intended to limit the present invention to a specific disclosed form. It should be understood that the scope of the present invention includes all modifications, equivalents and substitutes included in the spirit and technical scope of the present invention.

### Example 1

### Preparation of Bio-Derived Nanovesicles for Membrane Protein Delivery

The bio-derived nanovesicles for membrane protein delivery of the present invention were produced through the following major steps: (1) selection of immune-regulating protein or targeting protein candidates, (2) establishment of membrane protein expression cell lines, (3) induction and separation of plasma membrane blebs and (4) production and separation of nanovesicles using size control technology.

### 1-1. Bleb Production Protocol Using DTT and PFA

A solution including 10 mM HEPES, 150 mM NaCl, and 2 mM CaCl₂ dissolved in Auto DW was prepared, and the pH was adjusted to 7.4. Then, it was filtered through a 0.22 μm bottle top filter. The prepared vesicle solution can be stored at 4° C. for about 6 months. The 1M DTT stock was prepared by dissolving DTT powder to a concentration of 1 M, dispensing it into the desired amount, and storing it at −20° C. (20 μL DTT treatment based on 150 mm 1 dish).

Cells were prepared at a density of about 90%. Two tubes were prepared, one containing 10 mL of the vesicle solution, and the other containing 10 mL of the vesicle solution, 20 μL of 1 M DTT and 180 μL of 4% paraformaldehyde (PFA). The cells were taken out of the incubator and washed once with the vesicle solution, and the solution containing DTT and 4% PFA was added to the vesicle solution. In this case, the cells were added very carefully such that they did not fall off. After incubating at 37° C. for 4 hours, the solution was collected and centrifuged at 500 g for 5 minutes to remove the cells. Only the liquid was transferred to another tube and centrifuged at 3,000 g for 1 hour. The liquid was aspirated and removed, and the pellet was dissolved in 1 mL of PBS.

### 1-2. Bleb Production Protocol Using NEM

The bleb was produced in the same manner as in Example 1-1, except that 100 μL NEM was used instead of DTT and PFA.

### 1-3. Reduction of Bleb Size Using Extrusion

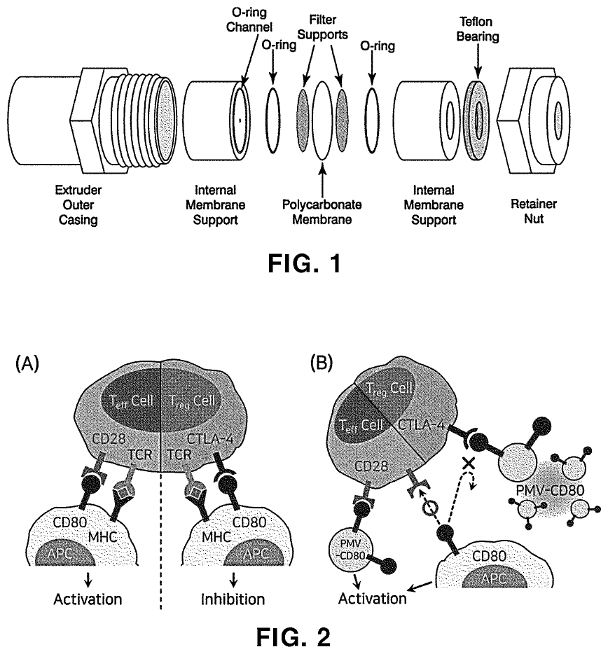

After producing the bleb according to the protocol of Example 1-1 or 1-2, extrusion was performed to reduce the size of the bleb. The structure of the extruder used is as shown in FIG. 1.

The solution including the produced bleb was centrifuged at 500 g for 5 minutes to remove cell debris, and centrifuged at 3,000 g for 1 hour. The pre-cleaned bleb was suspended in PBS (filtered through a 200 nm filter) at a concentration of 100 μg/mL to 1 mg/mL. Ethanol was first added to one syringe and washed 3 to 4 times. After removing the ethanol, PBS was added to one syringe and washed 3 to 4 times or more by passing it through. Herein, when removing PBS, half of the PBS was left in the syringe and it was washed by spraying a little PBS on the syringe needle connection part. Before putting the prepared bleb suspension, the front part of the syringe was locked well with tweezers. The pellet dissolved in PBS was put in the syringe, passed through the filter 11 times (once for each side) and transferred to another tube.

### 1-4. Reduction of Bleb Size Using Spray

After producing bleb according to the protocol of Example 1-1 or 1-2, air spray was performed in the following way to reduce the size.

The solution including the produced bleb was centrifuged at 500 g for 5 minutes to remove cell debris, and centrifuged at 3,000 g for 1 hour. The pre-cleaned bleb was suspended in PBS (filtered through a 200 nm filter) at a concentration of 100 μg/mL to 1 mg/mL. After assembling the airbrush (inner diameter 150 μm), ethanol (filtered through a 200 nm filter) was sprayed 1 mL at a pressure of 4 bar and washed 1 to 5 times. The washed airbrush was sprayed with 1 mL of PBS (filtered through a 200 nm filter) and washed 1 to 5 times. The prepared bleb suspension was added to the washed airbrush and sprayed once, and then centrifuged at 1,000 g for 20 minutes to separate cell debris.

### 1-5. Reduction of Bleb Size Using Ultrasound

After producing blebs according to the protocol of Example 1-1 or 1-2, ultrasound treatment was performed in the following manner to reduce the size.

The solution including the produced bleb was centrifuged at 500 g for 5 minutes to remove cell debris, and then centrifuged at 3,000 g for 1 hour. The pre-cleaned blebs were suspended in PBS (filtered through a 200 nm filter) at a concentration of 100 μg/mL to 1 mg/mL. The sonicator was placed in 20 mL of ethanol (filtered through a 200 nm filter) and washed 1 to 5 times for 30 seconds at 50 to 500 W. Then, the sonicator was placed in 20 mL of distilled water (filtered through a 200 nm filter) and washed 1 to 5 times for 30 seconds at 50 to 500 W. The prepared bleb suspension was sonicated at 50 to 500 W for 30 seconds and then stabilized on ice for 30 seconds, and this process was repeated 1 to 5 times.

### 1-6. Separation and Purification of Nanovesicles

The ultra tube was prepared by placing the same in hydrogen peroxide water in advance. The tube was washed twice with Auto DW, and 1 mL of opti-prep 10% and opti-prep 2% were stacked. The sample was stacked on top, and the remaining volume was filled with PBS. Ultracentrifugation was performed at 100,000 g for 2 hours. Samples were collected between opti-prep 10% and opti-prep 2%.

### Example 2

### Optimization of Nanovesicle Preparation Method

In order to derive an optimized nanovesicle preparation method, CD80, which is a T cell co-stimulatory molecule that induces immune action as an expressed protein, was selected as a membrane protein, and the particle size, particle concentration, protein yield and protein expression rate of nanovesicles prepared by varying the type of a bleb-inducing agent and the bleb size reduction method were comprehensively evaluated.

### 2-1. Cell Culture and Stable Cell Line Establishment

It was intended to induce T cell activation and suppression by using CD80 (FIG. 2). To this end, a cell line overexpressing CD80 fluorescence-labeled GFP on the cell membrane was established as follows to visualize the signaling protein.

HEK293T cell lines were cultured in Dulbecco's modified Eagle's medium (DMEM; Gibco, 12100046) supplemented with 1000 (v/v) fetal bovine serum (FBS; Gibco, 12483020) and antibiotic-antimycotic solution (Gibco, 15240062). Cells were maintained in a humidified incubator at 37° C. including 5% carbon dioxide (CO₂). The CD80-GFP encoding plasmid was purchased from Sino Biological (Sino Biological, HG10698-ACG, MG50446-ACG).

The nucleotide sequences of human CD80GFP and mouse CD80GFP are shown in Table 1. In each base sequence, the linker is indicated in bold, and GFP is indicated in underline.

TABLE 1

| | | SEQ ID |
|---|---|---|
| | Nucleotide Sequence | NO. |
| Human | ATGGGCCACACACGGAGGCAGGGAACATCACCATCCAAGTG | 1 |
| CD80GFP | TCCATACCTCAATTTCTTTCAGCTCTTGGTGCTGGCTGGTCTT | |
| | TCTCACTTCTGTTCAGGTGTTATCCACGTGACCAAGGAAGTG | |
| | AAAGAAGTGGCAACGCTGTCCTGTGGTCACAATGTTTCTGTT | |
| | GAAGAGCTGGCACAAACTCGCATCTACTGGCAAAAGGAGAA | |
| | GAAAATGGTGCTGACTATGATGTCTGGGGACATGAATATATGG | |
| | CCCGAGTACAAGAACCGGACCATCTTTGATATCACTAATAACC | |
| | TCTCCATTGTGATCCTGGCTCTGCGCCCATCTGACGAGGGCAC | |
| | ATACGAGTGTGTTGTTCTGAAGTATGAAAAAGACGCTTTCAA | |
| | GCGGGAACACCTGGCTGAAGTGACGTTATCAGTCAAAGCTG | |
| | ACTTCCCTACACCTAGTATATCTGACTTTGAAATTCCAACTTCT | |
| | AATATTAGAAGGATAATTTGCTCAACCTCTGGAGGTTTTCCAG | |
| | AGCCTCACCTCTCCTGGTTGGAAAATGGAGAAGAATTAAATG | |
| | CCATCAACACAACAGTTTCCCAAGATCCTGAAACTGAGCTCT | |
| | ATGCTGTTAGCAGCAAACTGGATTTCAATATGACAACCAACC | |
| | ACAGCTTCATGTGTCTCATCAAGTATGGACATTTAAGAGTGAA | |
| | TCAGACCTTCAACTGGAATACAACCAAGCAAGAGCATTTTCC | |
| | TGATAACCTGCTCCCATCCTGGGCCATTACCTTAATCTCAGTA | |
| | AATGGAATTTTTGTGATATGCTGCCTGACCTACTGCTTTGCCC | |
| | CAAGATGCAGAGAGAGAAGGAGGAATGAGAGATTGAGAAG | |
| | GGAAAGTGTACGCCCTGTAGGGGGTGGAGGCTCTGTGAGC | |
| | AAGGGCGAGGAGCTGTTCACCGGGGTGGTGCCCATCCTGGT | |
| | CGAGCTGGACGGCGACGTAAACGGCCACAAGTTCAGCGTGT | |
| | CCGGCGAGGGCGAGGGCGATGCCACCTACGGCAAGCTGACC | |
| | CTGAAGTTCATCTGCACCACCGGCAAGCTGCCCGTGCCCTGG | |
| | CCCACCCTCGTGACCACCCTGACCTACGGCGTGCAGTGCTTC | |
| | AGCCGCTACCCCGACCACATGAAGAAGCACGACTTCTTCAAG | |
| | TCCGCCATGCCCGAAGGCTACGTCCAGGAGCGCACCATCTTC | |
| | TTCAAGGACGACGGCAACTACAAGACCCGCGCCGAGGTGAA | |
| | GTTCGAGGGCGACACCCTGGTGAACCGCATCGAGCTGAAGG | |
| | GCATCGACTTCAAGGAGGACGGCAACATCCTGGGGCACAAG | |
| | CTGGAGTACAACTACAACAGCCACAACGTCTATATCATGGCC | |
| | GACAAGCAGAAGAACGGCATCAAGGCTAACTTCAAGGTTCG | |
| | CCACAACATCGAGGACGGCAGCGTGCAGCTCGCCGACCACT | |
| | ACCAGCAGAACACCCCCATCGGCGACGGCCCCGTGCTGCTG | |
| | CCCGACAACCACTACCTGAGCACCCAGTCCGCCCTGAGCAA | |
| | AGACCCCAACGAGAAGCGCGATCACATGGTCCTGCTGGAGTT | |
| | CGTGACCGCCGCCGGGATCACTCTCGGCATGGACGAGCTGTA | |
| | CAAGTAA | |
| Mouse | ATGGCTTGCAATTGTCAGTTGATGCAGGATACACCACTCCTCA | 2 |
| CD80GFP | AGTTTCCATGTCCAAGGCTCATTCTTCTCTTTGTGCTGCTGAT | |
| | TCGTCTTTCACAAGTGTCTTCAGATGTTGATGAACAACTGTCC | |
| | AAGTCAGTGAAAGATAAGGTATTGCTGCCTTGCCGTTACAAC | |
| | TCTCCTCATGAAGATGAGTCTGAAGACCGAATCTACTGGCAA | |
| | AAACATGACAAAGTGGTGCTGTCTGTCATTGCTGGGAAACTA | |
| | AAAGTGTGGCCCGAGTATAAGAACCGGACTTTATATGACAAC | |
| | ACTACCTACTCTCTTATCATCCTGGGCCTGGTCCTTTCAGACC | |
| | GGGGCACATACAGCTGTGTCGTTCAAAAGAAGGAAAGAGGA | |
| | ACGTATGAAGTTAAACACTTGGCTTTAGTAAAGTTGTCCATCA | |
| | AAGCTGACTTCTCTACCCCCAACATAACCGAGTCTGGAAACC | |
| | CATCTGCAGACACTAAAAGGATTACCTGCTTTGCTTCCGGGG | |
| | GTTTCCCAAAGCCTCGCTTCTCTTGGTTGGAAAATGGAAGAG | |
| | AATTACCTGGCATCAATACGACAATTTCCCAGGATCCTGAATC | |
| | TGAATTGTACACCATTAGTAGCCAACTAGATTTCAATACGACT | |
| | CGCAACCACACCATTAAGTGTCTCATTAAATATGGAGATGCTC | |
| | ACGTGTCAGAGGACTTCACCTGGGAAAAACCCCCAGAAGAC | |
| | CCTCCTGATAGCAAGAACACACTTGTGCTCTTTGGGGCAGGA | |
| | TTCGGCGCAGTAATAACAGTCGTCGTCATCGTTGTCATCATCA | |
| | AATGCTTCTGTAAGCACAGAAGCTGTTTCAGAAGAAATGAGG | |
| | CAAGCAGAGAAACAAACAACAGCCTTACCTTCGGGCCTGAA | |
| | GAAGCATTAGCTGAACAGACCGTCTTCCTTGGGGGTGGAGG | |
| | CTCTGTGAGCAAGGGCGAGGAGCTGTTCACCGGGGTGGTGC | |
| | CCATCCTGGTCGAGCTGGACGGCGACGTAAACGGCCACAAG | |
| | TTCAGCGTGTCCGGCGAGGGCGAGGGCGATGCCACCTACGG | |
| | CAAGCTGACCCTGAAGTTCATCTGCACCACCGGCAAGCTGCC | |
| | CGTGCCCTGGCCCACCCTCGTGACCACCCTGACCTACGGCGT | |
| | GCAGTGCTTCAGCCGCTACCCCGACCACATGAAGAAGCACG | |
| | ACTTCTTCAAGTCCGCCATGCCCGAAGGCTACGTCCAGGAGC | |
| | GCACCATCTTCTTCAAGGACGACGGCAACTACAAGACCCGCG | |
| | CCGAGGTGAAGTTCGAGGGCGACACCCTGGTGAACCGCATC | |
| | GAGCTGAAGGGCATCGACTTCAAGGAGGACGGCAACATCCT | |
| | GGGGCACAAGCTGGAGTACAACTACAACAGCCACAACGTCT | |
| | ATATCATGGCCGACAAGCAGAAGAACGGCATCAAGGCTAACT | |
| | TCAAGGTTCGCCACAACATCGAGGACGGCAGCGTGCAGCTC | |
| | GCCGACCACTACCAGCAGAACACCCCCATCGGCGACGGCCC | |
| | CGTGCTGCTGCCCGACAACCACTACCTGAGCACCCAGTCCGC | |
| | CCTGAGCAAAGACCCCAACGAGAAGCGCGATCACATGGTCC | |
| | TGCTGGAGTTCGTGACCGCCGCCGGGATCACTCTCGGCATGG | |
| | ACGAGCTGTACAAGTAA | |

HEK293T cells were transfected with plasmids by using Lipofectamine 3000 reagent (Invitrogen, L3000015). In order to establish a stable HEK293T cell line expressing CD80GFP on the plasma membrane, antibiotic selection was performed on the transfected cells by using 100 μg/mL hygromycin B (Sigma, H3274). The cells that survived the antibiotic selection process were counted and cultured in a 96-well plate such that a single cell was present per well, and cell line candidates derived from a single cell were selected. Afterwards, the CD80GFP-positive cell line with the highest expression rate was established through flow cytometry.

### 2-2. Confirmation of Nanovesicle Concentration and Particle Size Distribution According to the Type of Bleb-Inducing Agent

In Example 2-1, the cell line overexpressing mouse CD80GFP was treated with different types of bleb-inducing agents as shown in Table 2 to prepare plasma membrane bleb-derived nanovesicles expressing CD80, and then, the nanovesicle particle concentration and particle size distribution according to the type of bleb-inducing agent were evaluated.

TABLE 2

| | Type of Bleb- | Method of Reducing |
|---|---|---|
| | Inducing Agent | Bleb Size |
| Preparation | NEM | Extrusion |
| Example 1 | | |
| Preparation | DTT and PFA | Extrusion |
| Example 2 | | |

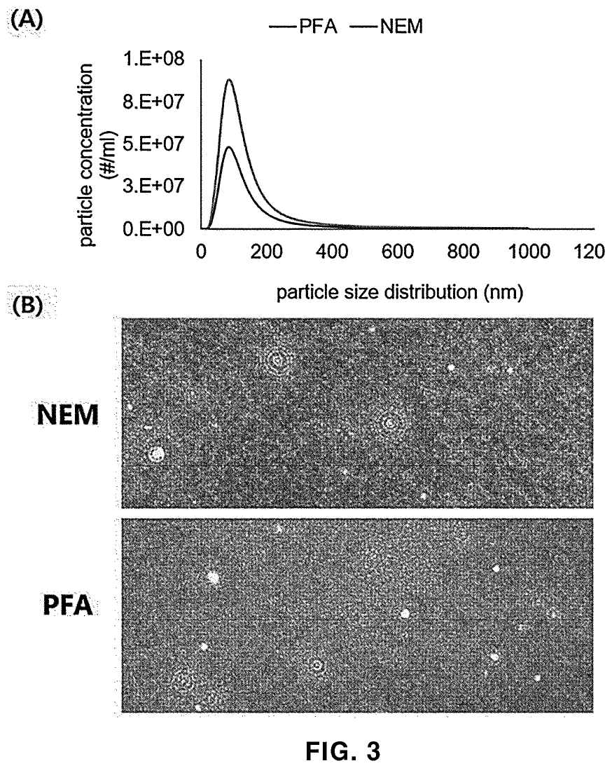

As shown in FIG. 3, the average particle sizes of the nanovesicles prepared by the method of Preparation Example 1 and the nanovesicles prepared by the method of Preparation Example 2 were similar at about 100 nm, but the particle concentration of the nanovesicles prepared by the method of Preparation Example 1 was twice that of the nanovesicles prepared by the method of Preparation Example 2, which confirmed that the use of NEM significantly improved the yield of nanovesicle preparation.

### 2-3. Confirmation of Nanovesicle Concentration and Particle Size Distribution According to the Method of Reducing Bleb Size

After preparing CD80-expressing plasma membrane bleb-derived nanovesicles by different methods of reducing bleb size in the cell line overexpressing mouse CD80GFP prepared in Example 2-1, the nanovesicle particle concentration and particle size distribution according to the method of reducing bleb size were evaluated.

TABLE 3

| | Type of Bleb- | Method of Reducing |
|---|---|---|
| | Inducing Agent | Bleb Size |
| Preparation | NEM | Extrusion |
| Example 3 | | |
| Preparation | NEM | Air Spray |
| Example 4 | | |
| Preparation | NEM | Ultrasonic Treatment |
| Example 5 | | |

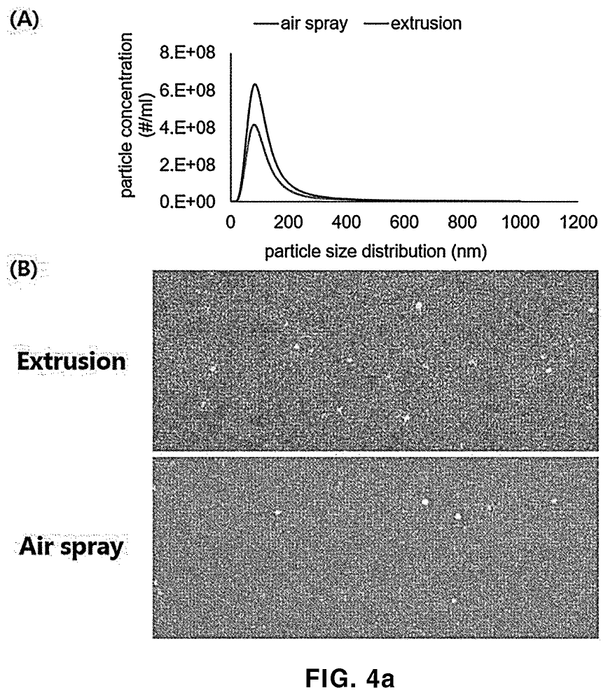

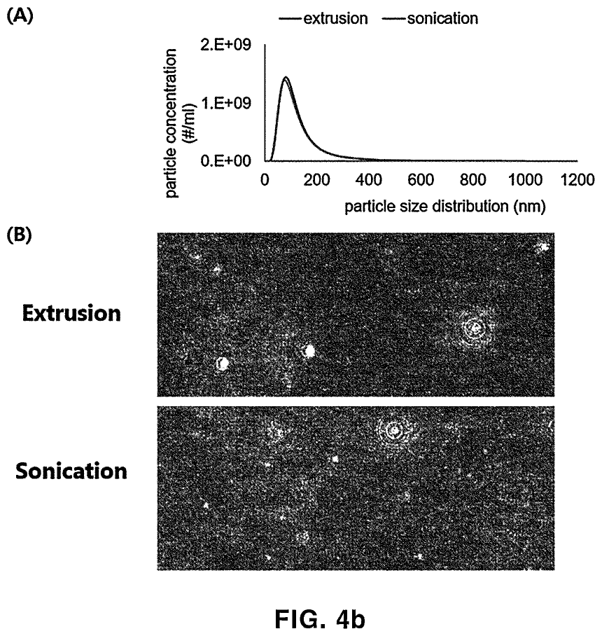

As shown in FIGS. 4a and 4b, the average particle size of the nanovesicles prepared by the methods of Preparation Examples 3 to 5 was similar at about 100 nm, and the particle concentrations of the nanovesicles prepared by the methods of Preparation Examples 3 and 5 were also confirmed to be similar. The nanovesicles prepared by the method of Preparation Example 4 had a relatively lower particle concentration than the nanovesicles prepared by the method of Preparation Example 3, but it was confirmed that there was no problem in preparing normal nanovesicles.

### 2-4. Confirmation of Protein Yield of Nanovesicles According to the Type of Bleb-Inducing Agent

After preparing plasma membrane bleb-derived nanovesicles expressing mouse CD80GFP in the same manner as in Example 2-2, the yield and expression rate of the expressed CD80 protein were evaluated.

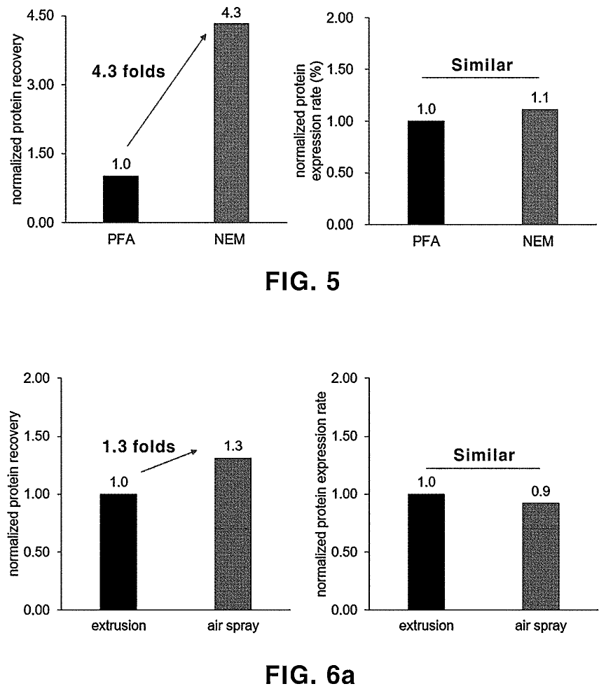

As shown in FIG. 5, there was no significant difference in the CD80 protein expression rates of the nanovesicles prepared by the methods of Manufacturing Examples 1 and 2, respectively, but the CD80 protein yield of the nanovesicles prepared by the method of Preparation Example 1 was about 4.3 times higher than that of the nanovesicles prepared by the method of Preparation Example 2, which confirmed that using NEM as a bleb-inducing agent significantly improved the protein yield.

### 2-5. Confirmation of Protein Yield of Nanovesicles According to the Method of Reducing Bleb Size

After preparing plasma membrane bleb-derived nanovesicles overexpressing mouse CD80GFP in the same manner as in Example 2-3, the yield and expression rate of the expressed CD80 protein were evaluated.

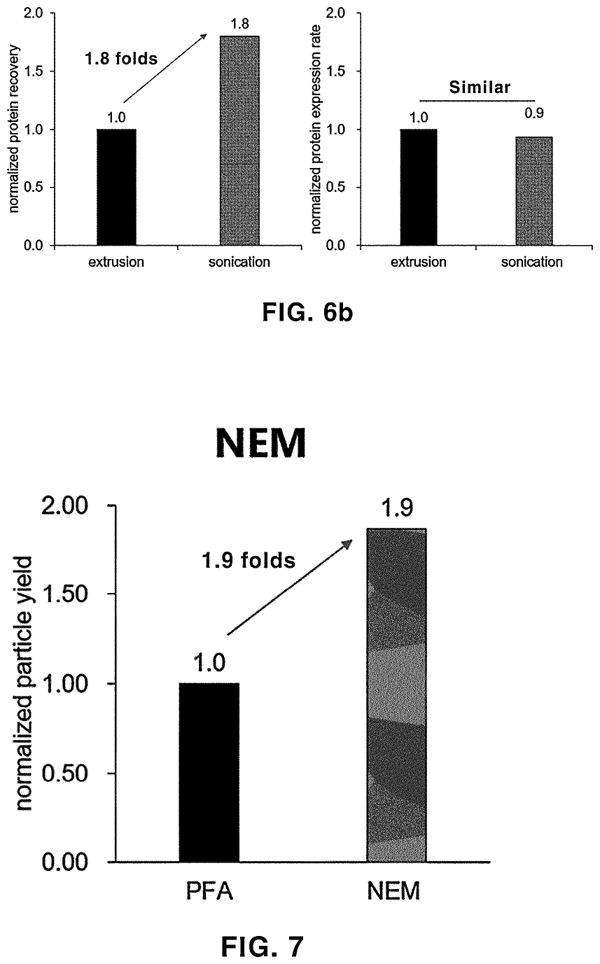

As shown in FIGS. 6a and 6b, there was no significant difference in the CD80 protein expression rates of the nanovesicles prepared by the methods of Preparation Examples 3 to 5, respectively, and the CD80 protein yield was confirmed to be high in the order of Preparation Example 3<Preparation Example 4<Preparation Example 5.

### 2-6. Confirmation of the Number of Nanovesicle Particles According to the Type of Bleb-Inducing Agent

After preparing nanovesicles derived from plasma membrane blebs that overexpress mouse CD80GFP in the same manner as in Example 2-2, the number of nanovesicle particles was evaluated.

As shown in FIG. 7, the number of particles of nanovesicles prepared by the method of Preparation Example 1 was approximately 1.9 times higher than the number of particles of nanovesicles prepared by the method of Preparation Example 2, which confirmed that using NEM as a bleb-inducing agent significantly improved the yield of nanovesicle preparation.

### 2-7. Confirmation of the Number of Nanovesicle Particles According to the Method of Reducing Bleb Size

After preparing nanovesicles derived from plasma membrane blebs that overexpress mouse CD80GFP in the same manner as in Example 2-3, the number of nanovesicle particles was evaluated.

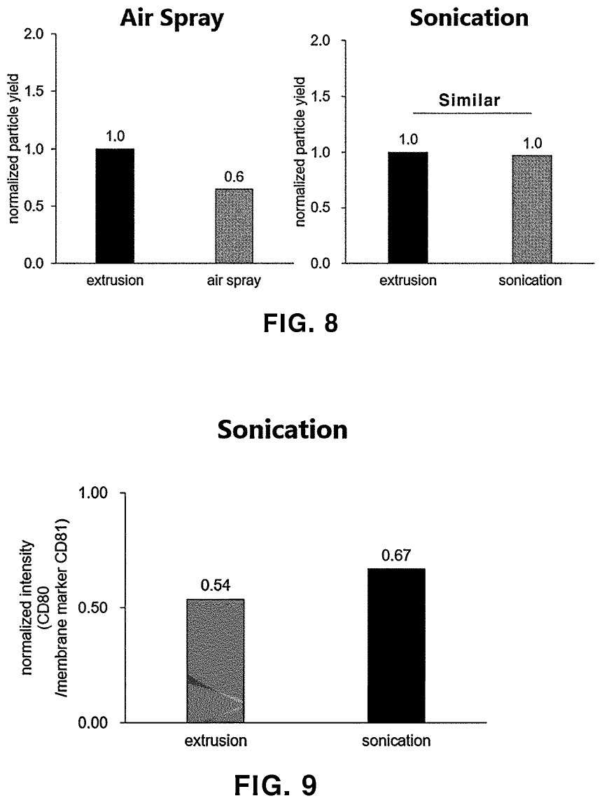

As shown in FIG. 8, the number of particles of nanovesicles prepared by the methods of Preparation Examples 3 and 5 were confirmed to be similar. Nanovesicles prepared by the method of Preparation Example 4 had a relatively smaller particle count than nanovesicles prepared by the method of Preparation Example 3, but it was confirmed that there was no problem in preparing normal nanovesicles.

### 2-8. Confirmation of Protein Expression Amount According to the Method of Reducing Bleb Size

After preparing plasma membrane bleb-derived nanovesicles overexpressing mouse CD80GFP in the same manner as in Example 2-3, the amount of CD80 protein expressed on the nanovesicle membrane was evaluated.

As a result, as shown in FIG. 9, nanovesicles prepared by the method of Preparation Example 3 had a relatively lower amount of CD80 protein expressed on the nanovesicle membrane compared to nanovesicles prepared by the method of Preparation Example 5, but it was confirmed that there was no problem in preparing normal nanovesicles.

### Example 3

### Preparation of Plasma Membrane Bleb-Derived Nanovesicles Expressing CD80

### 3-1. Flow Cytometry

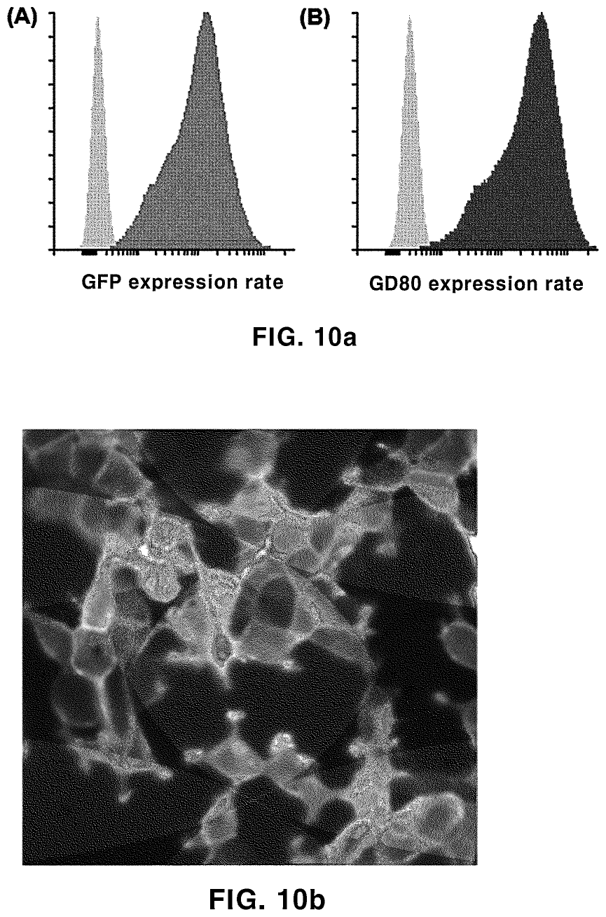

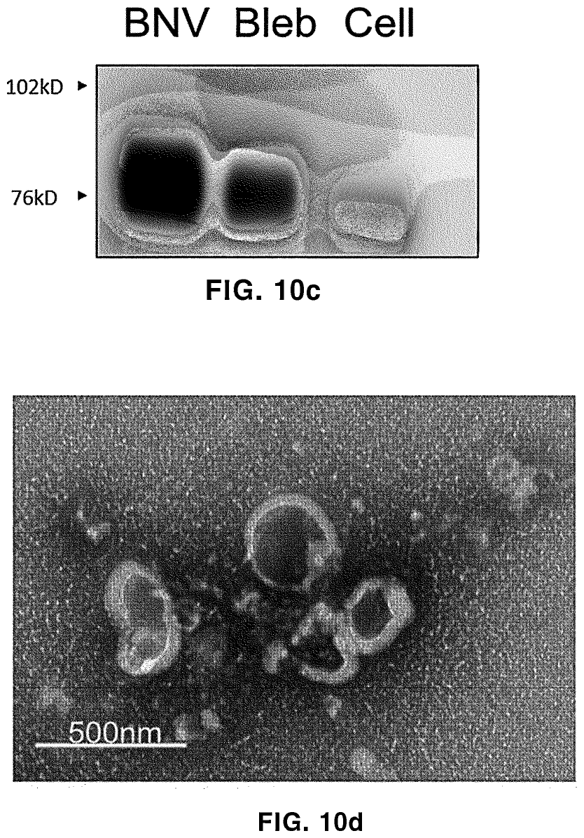

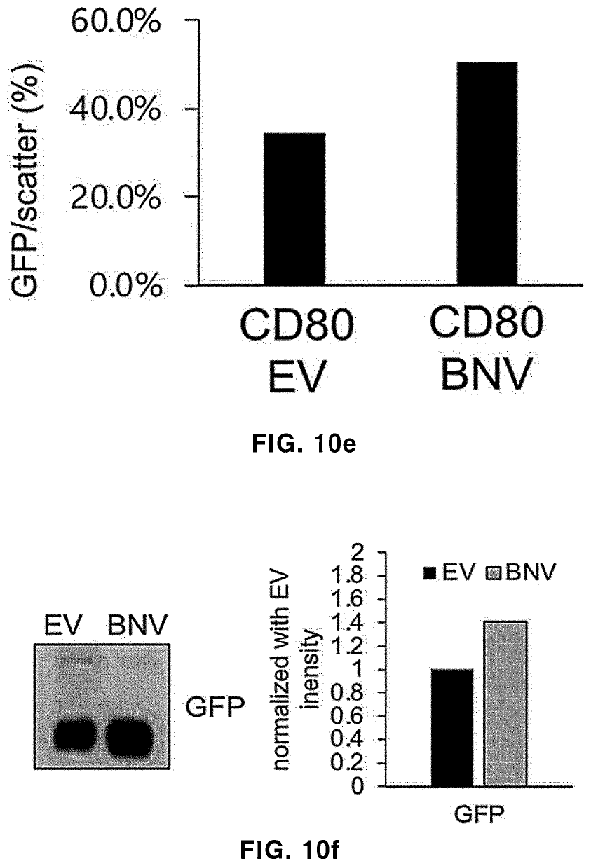

The CD80 signal and GFP fluorescence signal of the established cell line expressing the membrane protein in the form of CD80 conjugated to the C-terminus of GFP, which was prepared in Example 2-1, were analyzed by using a flow cytometer. Since the cell line expressed CD80GFP by transfection into the HEK293T cell line, the non-transfected HEK293T (WT) cell line was shown in gray as a control (left graph in (A) and (B) of FIG. 10a). The GFP signal bound to the C-terminus of CD80 was shown in green (right graph in (A) of FIG. 10a), and the signal of CD80 visualized using the CD80 antibody conjugated to APC fluorescence was shown in red (right graph in (B) of FIG. 10a). The x-axis of each graph represented the intensity of the fluorescence signal, and the y-axis represented the number of cells. As shown in FIG. 10a, the CD80GFP expressing cell line overexpressed CD80 compared to the wild type (WT) HEK293T cell line, and it was confirmed that the signal of this CD80 was visualized through GFP.

### 3-2. Induction and Separation of Plasma Membrane Blebs

The stable HEK293T cell line overexpressing the GFP fluorescence-labeled human CD80 prepared in Example 2-1 on the cell membrane was cultured in a 150 mm TC-treated culture dish (Corning, 430599). When the cells reached approximately 80% confluence, they were washed with basic buffer (10 mM HEPES, 150 mM NaCl, 2 mM CaCl₂), pH 7.4) and incubated in a humidified incubator at 37° C. and 5% CO₂ with bleb-inducing buffer (2 mM NEM dissolved in basic buffer). The supernatant including plasma membrane blebs was centrifuged at 500×g for 10 minutes to remove dead cells. The resulting supernatant was further centrifuged at 3,000×g for 30 minutes at room temperature to pellet the blebs. The pellet was gently resuspended in sterile PBS. Some blebs were used for fluorescence plate reader and microscopic analysis, while others were subjected to sequential extrusion for size reduction.

### 3-3. Generation and Isolation of Nanovesicles Using Size-Controlled Technology

The prepared blebs were sequentially extruded by using a mini-extruder set (Avanti Polar Lipids, 610000) equipped with 1000, 400 and 200 nm track-etched polycarbonate membrane filters (Avanti Polar Lipids, 610010, 610007, and 610006). After each extrusion step, a small amount of extrudate was saved for DLS analysis and NTA. The final extrudate (B200) was centrifuged at 100,000×g for 2 hours at 37° C. to pellet VNV. Next, the pellet was then resuspended in PBS for VNV characterization or stored at −80° C. for subsequent experiments.

### 3-4. Analysis of the Location of CD80GFP Protein Expression in Cell Lines Using Immunochemical Cell Imaging Analysis

Immunochemical cell imaging analysis was performed to identify the subcellular locations of CD80 signals and GFP fluorescence signals in cell lines expressing membrane proteins in which GFP was linked to the C-terminus of CD80. The GFP signal of the CD80GFP cell line is shown in green (488 nm excitation), the CD80 signal is shown in red (561 nm excitation), and the nuclear signal (405 nm excitation) is shown in blue. The images of each fluorescence channel were taken and merged by using ImageJ software.

For immunochemical cell imaging analysis, 10⁴ cells were treated per 6-well plate and cultured until 50% confluence. Afterwards, 4% PFA was used to fix the cells, and 3% BSA was used to block the cells at room temperature for 1 hour to prevent non-specific binding. The cells were treated with the primary antibody (Biolegend, cat. 305220) detecting CD80 at room temperature for 1 hour, and the washing process with PBS was repeated 3 times. Afterwards, the secondary antibody conjugated to AF546 fluorescence (Invitrogen, cat. A21123) was treated at room temperature for 1 hour, and the washing process with PBS was repeated 3 times. The signal of the nucleus in the cells was visualized by treating the cells for 10 minutes at room temperature with Hoechst 33342 dye, and then washing the same 3 times with PBS. The stained cells were analyzed through a fluorescence microscope (Olympus). As shown in FIG. 10b, most of the expressed CD80 and GFP signals were confirmed to be distributed along the plasma membrane rather than inside the cell.

### 3-5. Comparison of the Amount of CD80 Protein Through Western Blot Analysis

Western blot analysis was performed to determine the expression of the fusion protein and the structural denaturation in the produced cell line and BNV. In the case of the cell line, when the confluence of 50% in the 150 mm TC-treated culture dish was reached, the cells were harvested and centrifuged at 200 g for 5 minutes. The compressed material was dissolved by using RIPA buffer at 4° C. for 10 minutes, and then centrifuged at 10,000 g for 10 minutes to remove lipids. The supernatant (cell lysate) was harvested and used as a sample for Western blot. For BNV, the sample stored at −80° C. ([articles were separated by centrifugation after extruding 11 times through filters with pores of 1,000 nm->400 nm->200 nm in sequence) was dissolved and used.

The recovered cell lysate and BNV were measured for protein concentration by Bradford assay. Afterwards, 10 μg of each lysate per lane was separated on a 12% (w/v) polyacrylamide gel (GenDEPOT, a0418-050) by SDS-PAGE (Bio-Rad) under non-reducing conditions. Afterwards, the protein was transferred to a polyvinylidene fluoride (PVDF) membrane by using a wet transfer system (Bio-Rad). The Western blot membrane was gently shaken and incubated overnight at 4° C. in a blocking solution containing the primary antibody GFP (sc-9996). Then, the secondary antibody conjugated with HRP was incubated for 1 hour at room temperature and washed with TBS-T buffer. Afterwards, the membrane was incubated with a chemiluminescent substrate (Amersham, RPN2232), and the signal was captured by using a c-Digit Western blot scanner (LI-COR). The bands in the cropped images were detected under the same imaging conditions.

The amount of CD80 protein was determined by comparing the intensity of the bands in the molecular weight of the CD80 protein to which GFP was bound using the GFP antibody (CD80 protein molecular weight: 50-65 kDa, GFP protein molecular weight: ˜27 kDa, a band of about 70 kDa or more should be observed for GFP-bound CD80). As shown in FIG. 10c, the amount of CD80GFP protein of BNV was confirmed to be the most abundant compared to the same amount of protein, and the presence or absence of CD80GFP protein detection confirmed that CD80GFP did not significantly denature during the bleb induction and nano-processing (bleb size reduction process).

### 3-6. Structural Confirmation of Bleb-Derived Nanoparticles (BNV) Using Transmission Electron Microscopy

In order to visualize BNV, all samples were loaded onto formaldehyde/carbon-supported copper grids (Sigma, TEM-FCF300CU50) at room temperature for 1 minute. In order to enhance image contrast, the grids were negatively stained with Uranyless solution (EMS, 22409) for approximately 30 seconds. Subsequently, the grids were then dried overnight and imaged by using TEM (JOEL).

As shown in FIG. 10d, the blebs were reduced to an average diameter of less than 200 nm and were confirmed to be surrounded by a lipid bilayer.

### 3-7. Analysis of Individual Nanoparticles for Comparison of CD80GFP Expression Rates Between Extracellular Vesicles (EVs) and Bleb-Derived Nanoparticles (BNV)

Individual nanoparticle analysis was performed to compare the homogeneity of the prepared particles by confirming the distribution of CD80-expressing particles relative to the total particles. The number of particles with GFP signals compared to the total particles (scatter) of plasma membrane bleb-derived particles (BNV) and conventional extracellular vesicles (EV) was compared by using a fluorescence nanoparticle tracking analysis. The particle suspension was diluted with 200 nm-filtered PBS to create a particle suspension with a measurable concentration range and measured. The expression of CD80 GFP was visualized as a GFP fluorescence signal using a 488 nm laser.

As shown in FIG. 10e, it was confirmed that the bleb-derived nanoparticles (BNV) showed a homogeneous particle distribution that was approximately 1.5 times higher than that of conventional extracellular vesicles (EV).

### 3-8. Western Blot Analysis for Comparison of CD80GFP Expression Rates Between Extracellular Vesicles (EV) and Bleb-Derived Nanoparticles (BNV)

Ensemble analysis (Western blot) was performed to compare the amount of CD80 between cell-derived nanoparticles by confirming the amount of CD80GFP protein compared to the total protein amount. Western blot was performed through the same process as Example 3-4, and the lysate stored at −80° C. was loaded at 3 μg per lane. Similarly, the amount of CD80GFP protein was visualized by using GFP antibody (Santa Cruz, sc-9996). As shown in FIG. 10f, when it was normalized to the intensity of the band representing CD80GFP in EV, it was confirmed that approximately 1.5 times more CD80GFP protein was expressed in BNV compared to the same amount of protein.

### Example 4

Preparation of plasma membrane bleb-derived particles expressing fusion proteins including nanobodies

### 4-1. Fusion Protein Design and DNA Extraction

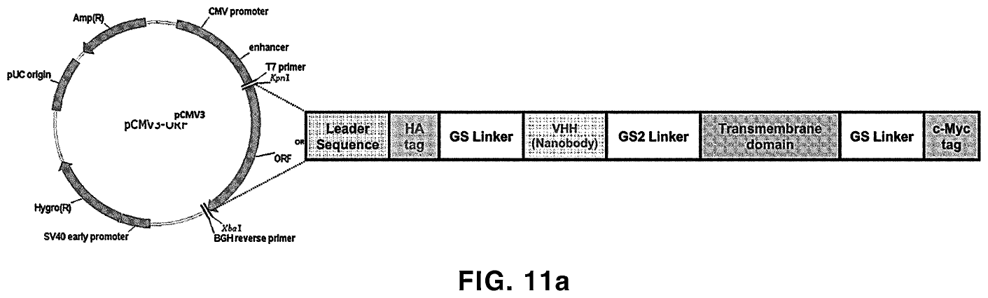

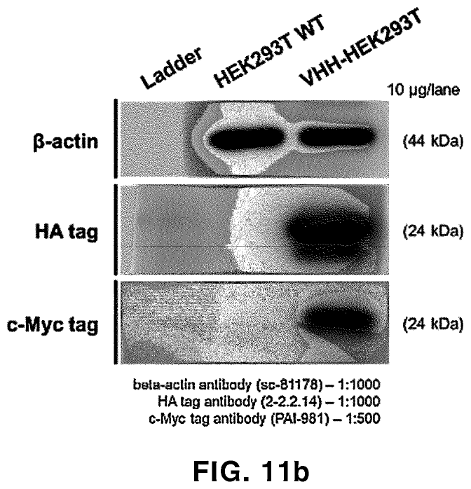

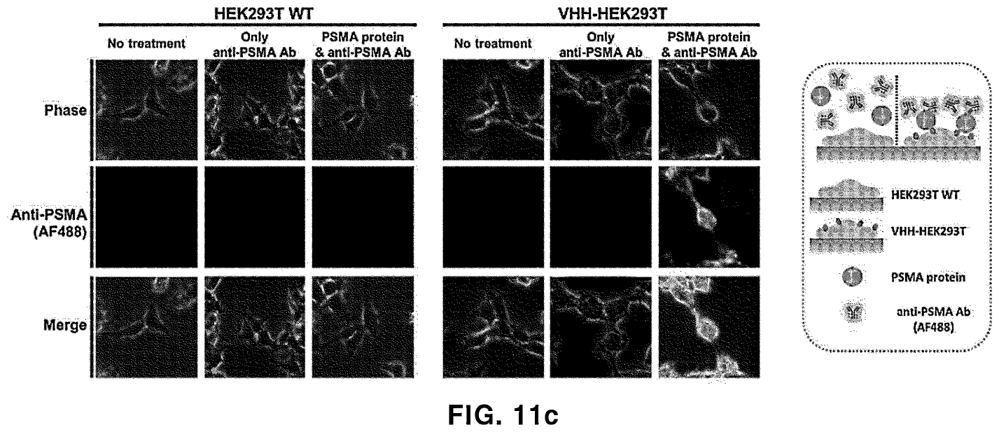

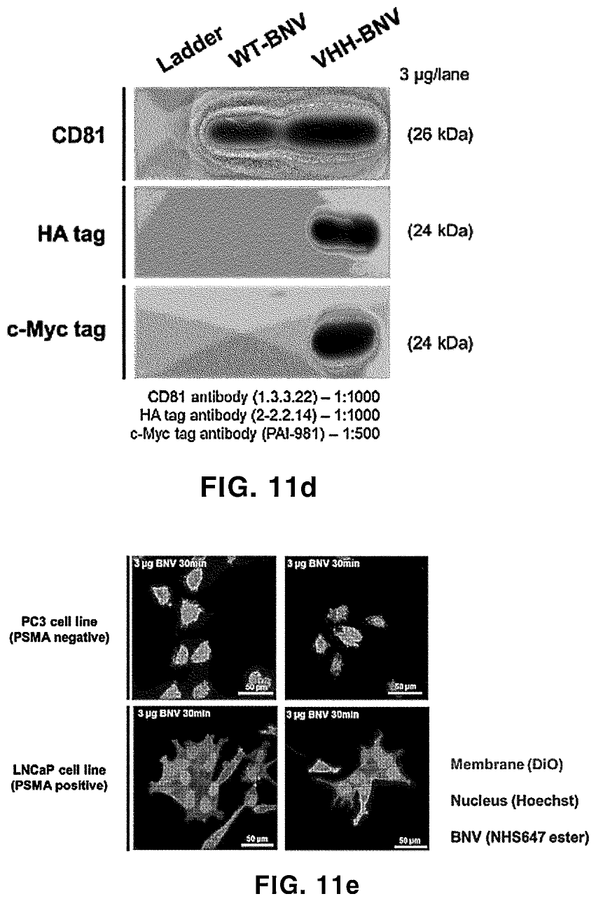

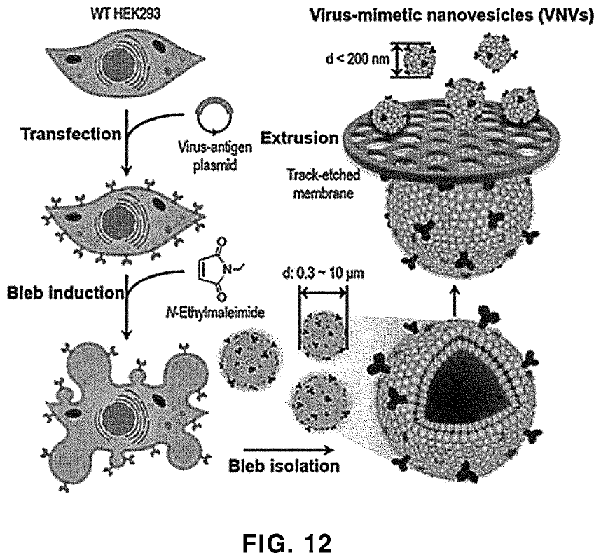

Plasma membrane bleb-derived particles (BNV) were generated from cell lines overexpressing fusion proteins including nanobodies for targeting, in order to target cancer. To this end, cell lines overexpressing nanobody fusion proteins expressed through a combination of a nanobody for targeting, a membrane protein for positioning the same on the cell membrane, a selected linker and a tag for labeling (tag selection) were established as follows (FIG. 11a).

The pCMV3-untagged plasmid vector (VG40588-UT) for constructing the fusion protein was purchased from Sino Biological. The sequence of the VHH antibody targeting prostate-specific membrane antigen (PSMA) was provided by RCSB PDB (PDB ID: 6XXN). The sequence of the membrane protein region of the integrin alpha1 (ITGA1) protein was provided by UniProt (UniProt ID:P56199). The DNA sequence of the designed fusion protein was commissioned to Bionics for gene synthesis and cloning.

TABLE 4

| | | SEQ ID |
|---|---|---|
| | Nucleotide Sequence | NO. |
| Fusion | GGTACCGCCACAATGCGGCTGCCCGCCCAGCTGCTGGGGCTG | 3 |
| Protein | CTGATGCTGTGGGTGCCCGGCAGCAGCGGCTACCCCTACGAT | |
| | GTGCCCGACTACGCCGGGGGCGGCGGGTCCCAGGTGCAGCT | |
| | GCAGGAGAGCGGGGGCGGCAGCGTGCAGGCCGGCGGGAGC | |
| | CTGAGGCTGAGCTGCACAGCCCCAGGGTACACCGACAGCAA | |
| | CTACTACATGAGCTGGTTCAGGCAGGCCCCTGGGAAGGAGCG | |
| | GGAGTGGGTGGCCGGGGTGAACACAGGCAGGGGCAGCACC | |
| | AGCTACGCCGACACCGTGAAGGGGCGGTTCACCATCAGCCA | |
| | GGATAACGCCAAGAACACCATGTTCCTGCAGATGAACAGCCT | |
| | GAAACCCGAGGACACAGCCATCTACTACTGCGCCGTGGCCGC | |
| | CTGCCACTTCTGCGACTCCCTTCCCAAGGGACAGGACGAGCA | |
| | GATCCTGTGGGGGCAGGGCACTCAGGTCACAGTGAGCAGCG | |
| | GGGGCGGCGGGAGCGGGGGGGGCGGGAGCCTGTGGGTCATC | |
| | CTGTTGAGCGCATTTGCTGGACTGTTGTTGTTGATGTTGCTTA | |
| | TTTTGGCTTTGTGGGGAGGGGGAGGGAGCGAGCAGAAGCTG | |
| | ATCTCCGAGGAGGATTTGTAATCTAGA | |

### 4-2. Cell Culture and Stable Cell Line Establishment

The HEK293T cell lines were obtained from the Korean Cell Line Bank (KCLB) and cultured in high glucose Dulbecco's modified Eagle's medium (H-DMEM; Gibco, 12800017) supplemented with 10% (v/v) fetal bovine serum (FBS; Gibco, 12484028) and antibiotic-antimycotic solution (Gibco, 15240062). The cells were maintained in a humidified incubator at 37° C. with 5% carbon dioxide (CO₂).

HEK293T cells were transfected with each plasmid by using Lipofectamine 3000 reagent (Invitrogen, L3000001). In order to establish stable HEK293T cell lines expressing the fusion protein performing a prostate tumor targeting function, antibiotic selection was performed on transfected cells by using 200 μg/mL hygromycin B (Sigma, H3274). Fusion protein-positive cells were sorted by using FACS (MoFlo Astrios EQ, Beckman Coulter).

Human prostate tumor cell lines, LNCaP-FGC cell lines and PC3 cell lines, were obtained from the Korean Cell Line Bank (KCLB) and cultured in RPMI 1640 (Gibco, 11875-093) medium supplemented with 10% (v/v) fetal bovine serum (FBS; Gibco, 12484028) and antibiotic-antimycotic solution (Gibco, 15240062). Cells were maintained in a humidified incubator at 37° C. with 5% carbon dioxide (CO₂).

### 4-3. Induction and Isolation of Plasma Membrane Blebs

Stable HEK293T cell lines were cultured in 150 mm TC-treated culture dishes (Corning, 430599). When the cells reached approximately 80% confluence, they were washed with abase buffer (10 mM HEPES, 150 mM NaCl, 2 mM CaCl₂), pH 7.4) and incubated with a bleb-induction buffer (2 mM NEM dissolved in basic buffer) in a humidified incubator at 37° C. with 5% CO₂. The supernatant containing the plasma membrane blebs was then subjected to centrifugation at 500×g for 10 minutes to eliminate dead cells. The resulting supernatant was further centrifuged at 3,000×g for 30 minutes at room temperature to pellet the blebs. The pellet was gently resuspended in sterile PBS. Some blebs were utilized for fluorescence plate reader and microscopic analyses, while others underwent serial extrusion for size reduction.

### 4-4. Generation and Isolation of Nanovesicles Using Size-Controlled Technique

The prepared blebs were sequentially extruded by using a mini-extruder set (Avanti Polar Lipids, 610000) equipped with 1000, 400 and 200 nm track-etched polycarbonate membrane filters (Avanti Polar Lipids, 610010, 610007, and 610006). After each extrusion step, a small amount of extrudate was saved for DLS analysis and NTA. The final extrudates (B200) were subjected to ultracentrifugation at 100,000×g for 2 hours at 37° C. to pellet VNVs. Next, the pellet was then resuspended in PBS for VNV characterizations or stored at −80° C. for subsequent animal experiments.

### Example 5

### Expression and Functional Evaluation of Fusion Protein

### 5-1. Western Blot Analysis

In order to determine the expression of the fusion protein in the cell line and BNV prepared in Example 4, Western blot analysis was performed in the same manner as in Example 3-4. The Western blot membrane was gently shaken and incubated overnight at 4° C. in a blocking solution including the following primary antibodies: 0-actin (Santa Cruz, sc-81178), CD81 (Santa Cruz, sc-7637), HA (Invitrogen, 26183) and c-Myc (Invitrogen, PAI-981). Subsequently, the membrane was then incubated with a chemiluminescent substrate (Amersham, RPN2232), and the signal was captured by using a c-Digit Western blot scanner (LI-COR). The bands in the cropped images were detected under the same imaging conditions.

As confirmed in FIGS. 11b and 11d, in the Western blot analysis, only the cell line including the fusion protein and the BNV showed strong bands for the HA tag and the c-Myc tag, indicating that the constructed cell line and the BNV produced from the cell line expressed the fusion protein well.

### 5-2. Fluorescent Antibody Analysis

In order to determine the function of the fusion protein in the constructed cell line and BNV, a fluorescent antibody analysis was performed.

In the case of the HEK293T cell line expressing the fusion protein, 104 cells were treated in a 6-well plate and then incubated to 30% confluence. The cells were fixed by using 2% PFA and blocked by using 3% BSA at room temperature for 1 hour. After reacting the cells with the PSMA antigen for 1 hour, the process of washing them cleanly with FPBS was repeated 3 times. On top of that, the HA tag antibody (Biolegend, 901509) was treated and reacted for 1 hour, and then, the process of washing thoroughly with FPBS was repeated 3 times. The cells that completed the process were subjected to fluorescent antibody analysis using a fluorescence microscope (Olympus).

As confirmed in FIG. 11c, fluorescence was observed only when the PSMA antigen and HA tag antibody were treated in the cell line expressing the fusion protein, indicating that the produced fusion protein smoothly performed the function of targeting PSMA on the cell.

In the case of BNV, it was stained in advance by using Alexa Fluor 488 NHS Ester (Invitrogen, A20000) dye for fluorescent antibody analysis. BNV, which reacted for 2 hours at 37° C., was separated from the dye through size exclusion chromatography (SEC). The separated BNV was counted through nanoparticle analysis, and then, experiments were conducted.

In order to determine the function of BNV, fluorescent antibody analysis was performed by using LNCaP cell lines and PC3 cell lines. As with the process using the HEK293T cell line, the blocking process was performed. DiI dye and Hoechst dye were used to stain the cell membrane and nucleus of the two cell lines, respectively. Afterwards, the dye-stained BNV was treated and reacted for 30 minutes, and then, the process of washing cleanly with FPBS was repeated three times. Each cell line that completed the process was subjected to fluorescence analysis using a confocal microscope.

As confirmed in FIG. 11e, only the LNCaP cell line that possessed the PSMA antigen showed a stained BNV fluorescence signal, indicating that the constructed fusion protein smoothly performed the function of targeting PSMA on BNV.

### Example 6

### Preparation of Enveloped Virus-Mimetic Nanovesicles

### 6-1. Cell Culture and Stable Cell Line Establishment

The HEK293 cell lines were obtained from the Korean Cell Line Bank (KCLB) and cultured in Dulbecco's modified Eagle's medium (DMEM; Gibco, 12100046) supplemented with 10% (v/v) fetal bovine serum (FBS; Gibco, 12483020) and antibiotic-antimycotic solution (Gibco, 15240062). Cells were maintained in a humidified incubator at 37° C. with 5% carbon dioxide (CO₂). The cytoplasmic GFP-encoding pCEP4 plasmid was kindly provided by Dr. Yong-Song KO, and the mCherry-mem-encoding plasmid was kindly provided by Catherine Berlot (Addgene plasmid #55779; http://n2t.net/addgene:55779; RRID: Addgene_55779). The Spike del (ERRS) encoding plasmid was provided by Zhaohui Qian (Addgene plasmid #145780; http://n2t.net/addgene:145780; RRID: Addgene_145780). Wild-type SARS-CoV-2 Spike- and Spike-GFP encoding plasmids were purchased from Sino Biological (Sino Biological, VG40589-UT and VG40590-ACG).

HEK293 cells were transfected with each plasmid by using Lipofectamine 3000 reagent (Invitrogen, L3000015). In order to establish stable HEK293 cell lines expressing cytoplasmic GFP and plasma membrane-localized mCherry, antibiotic selection was performed on the transfected cells by using 500 μg/mL G418 (Gibco, 10131027) and 250 μg/mL hygromycin B (Sigma, H3274). GFP- and mCherry-positive cells were sorted by using FACS (MoFlo Astrios EQ, Beckman Coulter).

### 6-2. Induction and Isolation of Plasma Membrane Blebs

Stable HEK293 cell lines were cultured in 150 mm TC-treated culture dishes (Corning, 430599). When cells reached approximately 80% confluence, they were washed with basic buffer (10 mM HEPES, 150 mM NaCl, 2 mM CaCl₂), pH 7.4) and cultured with bleb-inducing buffer (2 mM NEM dissolved in basic buffer) at 37° C. in a humidified incubator with 5% CO₂. The supernatant including plasma membrane blebs was centrifuged at 500×g for 10 minutes to remove dead cells. The resulting supernatant was further centrifuged at 3,000×g for 30 minutes at room temperature to pellet the blebs. The pellet was gently resuspended in sterile PBS. Some blebs were used for fluorescence plate reader and microscopic analysis, and others were subjected to serial extrusion for size reduction.

### 6-3. Generation and Isolation of Nanovesicles Using Size-Controlled Technology

Prepared blebs were sequentially extruded by using a mini-extruder set (Avanti Polar Lipids, 610000) equipped with 1000, 400 and 200 nm track-etched polycarbonate membrane filters (Avanti Polar Lipids, 610010, 610007, and 610006). After each extrusion step, a small amount of extrudate was saved for DLS analysis and NTA. The final extrudate (B200) was ultracentrifuged at 100,000×g for 2 hours at 37° C. to pellet VNV. The pellet was then resuspended in PBS for VNV characterization or stored at −80° C. for subsequent animal experiments.

### Example 7

### Confirmation of Optimal Conditions for Plasma Membrane Yield

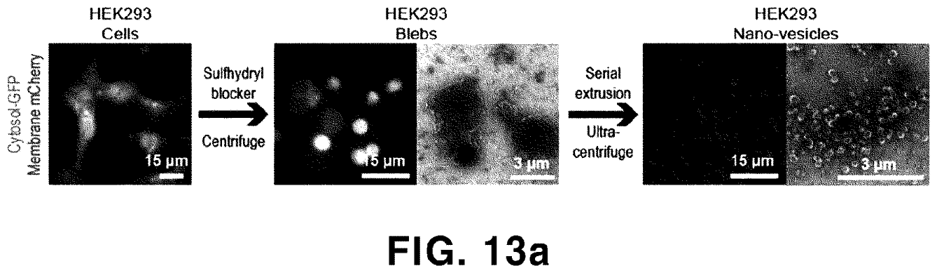

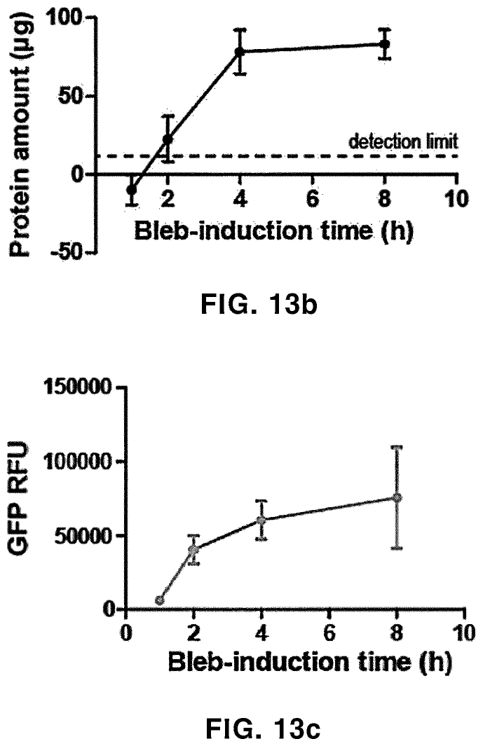

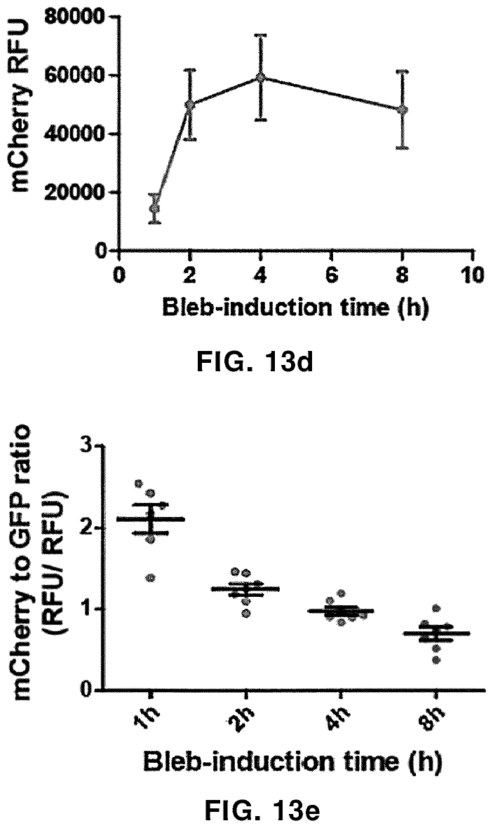

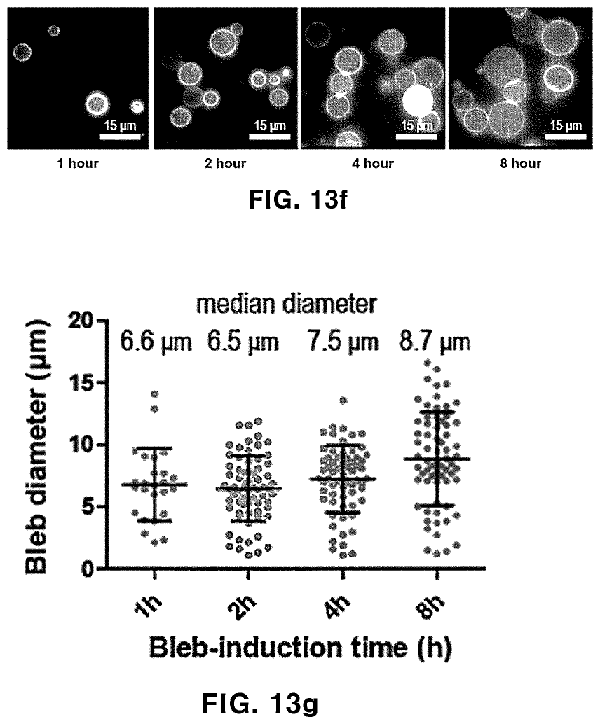

Since the present invention uses the plasma membrane of cells as a main material, it is important to use optimal conditions that maximize the yield of the plasma membrane while preventing other negative effects such as cell death in producing the plasma membrane blebs. Accordingly, in this example, factors affecting VNV production were investigated by using the reporter HEK293 cell line expressing cytoplasmic-GFP and plasma membrane-anchored mCherry prepared in Example 6 (FIG. 13a). The reporter cells were treated with a bleb-inducing solution for various times (1, 2, 4 and 8 hours, respectively) to determine the optimal incubation time for plasma membrane bleb production.

The optimal culture period was determined by quantifying the amount of blebs obtained at various incubation times, by considering total protein, cytoplasmic content (GFP expression) and plasma membrane content (indicated by mCherry expression). As confirmed in FIGS. 13b, 13c and 13d, all three indicators showed that the number of blebs increased up to 4 hours of incubation and reached the saturation point thereafter. As confirmed in FIG. 2e, as the incubation time increased, the ratio of mCherry to GFP content of the blebs decreased, indicating that the bleb size increased. In addition, as a result of examining the plasma membrane blebs with a fluorescence microscope, as confirmed in FIGS. 13f and 13g, the median diameter of the blebs increased as the incubation time increased.

The results showed that an incubation period longer than 4 hours did not significantly improve the bleb yield in terms of protein, cytoplasmic content and plasma membrane content, but rather increased the size of the generated blebs. Since an increase in the size of the blebs is not a feature required in the present invention, the plasma membrane blebs obtained by 4 hours of exposure were used.

### Example 8

### Control of the Size of Plasma Membrane Blebs

### 8-1. Nanovesicle Size Analysis

Dynamic light scattering analysis (Malvern, Zetasizer 3000HSA) and nanoparticle tracking analysis (Malvern, Nanosight) were performed on all extrudates. The extrudates were diluted 20-fold in filtered PBS, and each sample was measured five times in a 10-second run configuration. For NTA analysis, the extrudates were diluted 1,000 to 3,000 folds in filtered PBS to achieve a concentration of approximately 100 particles per imaging field. Samples were analyzed in six different fields, and the size and number of vesicles were calculated from the measurements.

### 8-2. Transmission Electron Microscopy (TEM)

In order to visualize the extrudates, all samples were loaded onto formaldehyde/carbon-supported copper grids (Sigma, TEM-FCF300CU50) at room temperature for 1 minute. The grids were negatively stained with Uranyless solution (EMS, 22409) for approximately 30 seconds to enhance the image contrast. The grids were then dried overnight and imaged by using TEM (JOEL).

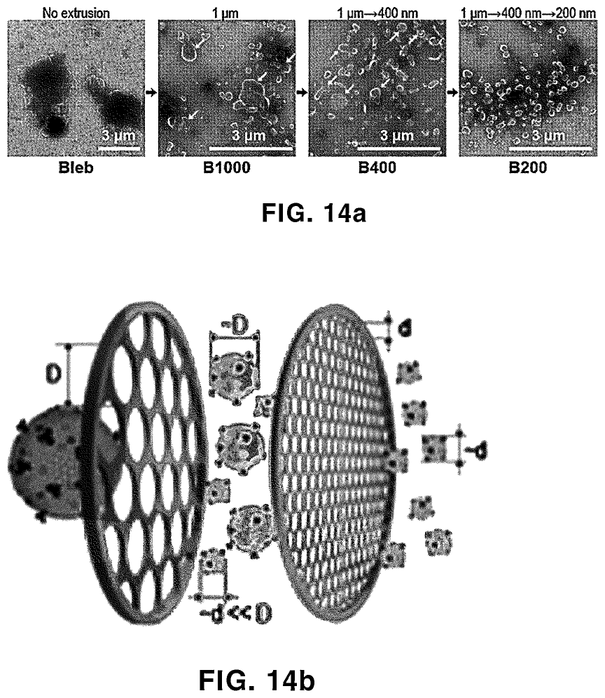

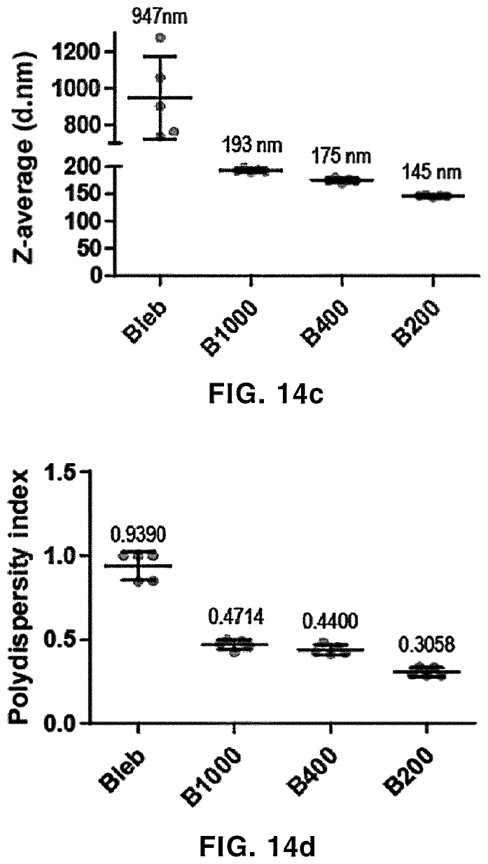

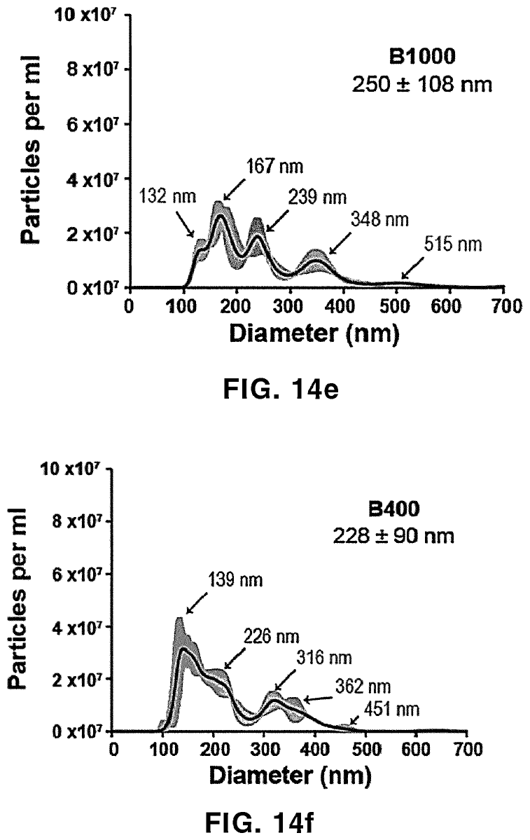

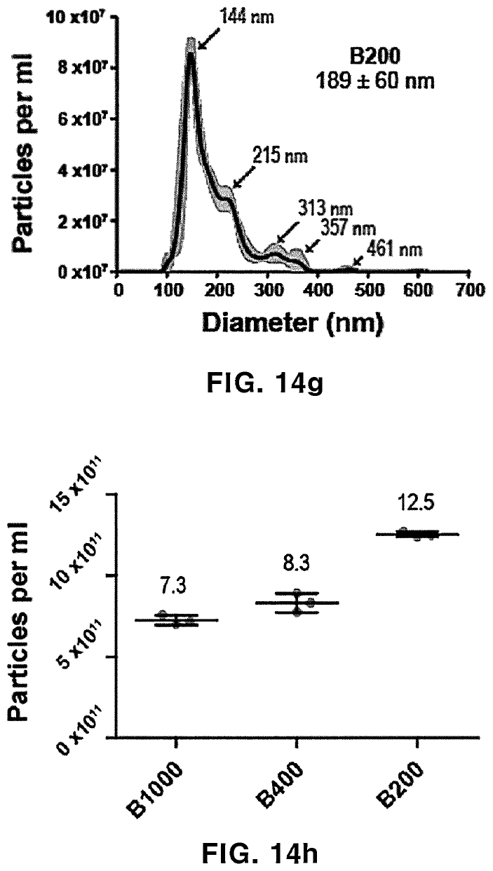

As confirmed in FIG. 14a, the blebs were extruded through multiple nanoporous membranes to reduce the average diameter to less than 200 nm, similar to the average diameter of the enveloped virus. Transmission electron microscopy images of blebs single-extruded through a 1,000 nm pore filter (B1000) and blebs double-extruded through 1,000 and 400 nm pore filters (B400) exhibited considerably diminished vesicle sizes compared to the original blebs. However, the extrudates indicated by the red arrows in FIG. 14a exhibited heterogeneous vesicle populations in terms of size, with larger vesicles having sizes paralleling those of the pores used for extrusion. Subsequent extrusion through a 200 nm pore filter (B200) yielded almost uniformly sized nanovesicles, with sizes of approximately 200 nm, as observed under TEM in FIG. 14a.

All extrudates underwent quantitative size analysis using dynamic light scattering and nanoparticle tracking analysis. The DLS results revealed an average diameter of less than 200 nm for all extrudates. However, as confirmed in FIGS. 14c and 14d, the polydispersity indices suggested that the vesicles within B1000 and B400 were too polydisperse to be assessed via DLS analysis. The polydispersity index of the B200 extrudate was approximately 0.3, indicating that the vesicles in B200 were comparatively monodisperse in size and resembled naturally occurring enveloped viruses.

The NTA results in FIG. 14g confirmed that the average diameter of the vesicles in the B200 was less than 200 nm, with a sharp peak diameter of 144 nm. In contrast, the NTA results for the other two extrudates in FIGS. 14e and 14f showed that the average diameter exceeded 200 nm and exhibited multiple broad peaks. In addition, as confirmed in FIG. 14h, the number of vesicles increased as the pore size diminished. By combining the qualitative analysis based on TEM images with quantitative analysis using DLS and NTA, it was observed that only the vesicles in the B200 extrudate achieved a sub-200 nm diameter, closely resembling the size of enveloped viruses.

The above results show that when the sequential extrusion method using multiple porous filters was used, nanovesicles with very uniform sizes could be obtained compared to when a single filter was used, and the sizes of the finally obtained nanovesicles were confirmed to have a very uniform size of less than 200 nm in all DLS, NTA and TEM.

### Example 9

### Analysis of the Plasma Membrane Content of Extruded Plasma Membrane-Derived Nanovesicles

### 9-1. Determination of the Plasma Membrane to Cytoplasm Ratio

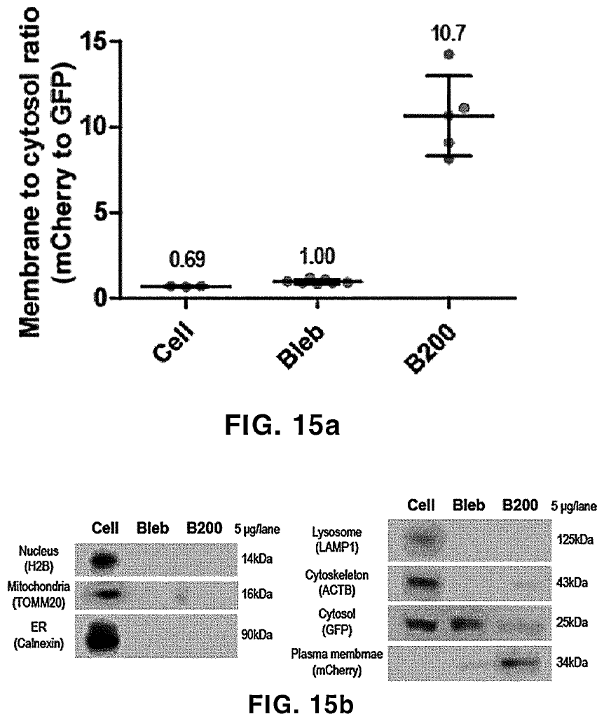

The composition of nanovesicles within the B200 extrudate was examined and compared with the composition of the original reporter cells and blebs. The relative plasma membrane content of the vesicles was quantified by measuring the ratio of plasma membrane-associated mCherry expression to cytoplasm-associated GFP expression. As a result, as confirmed in FIG. 15a, the ratio for B200 nanovesicles was approximately 10, which was substantially higher than the ratio of originating cells and blebs, which has a ratio of less than 1. The bleb-extruded nanovesicles exhibited only mCherry signals, with no discernible GFP signals, which is also supported by the microscopic observation results in FIG. 13a.

### 9-2. Western Blot Analysis

In order to investigate which cellular components could be transferred to plasma membrane blebs and bleb-extruded nanovesicles, Western blot was performed to detect protein markers associated with representative cellular organelles.

The equal amounts of proteins were lysed from cell lysates, vesicle lysates and VNV lysates by using RIPA (Radioimmunoprecipitation assay) buffer (Biosesang, P2002). Afterwards, 5 μg of each lysate per lane was separated on a 12% (w/v) polyacrylamide gel (GenDEPOT, a0418-050) by SDS-PAGE (Bio-Rad) under non-reducing conditions. The proteins were then transferred to polyvinylidene fluoride (PVDF) membranes using a wet transfer system (Bio-Rad). Western blot membranes were incubated overnight at 4° C. with gentle shaking in blocking solution containing the following primary antibodies: H2B (Cell Signaling, 12364), TGN38 (SCBT, SC165594), Calnexin (SCBT, SC11397), LAMP1 (SCBT, SC20011), ACTB (SCBT, SC81178), GFP (SCBT, SC9996) and DsRed (SCBT, SC390909). Subsequently, the membranes were incubated with a chemiluminescent substrate (Amersham, RPN2232), and signals were captured by using a c-Digit Western blot scanner (LI-COR). Bands in the cropped images were detected under the same imaging conditions.

As confirmed in FIG. 15b, cell lysate analysis showed strong bands for most organelle markers except for the plasma membrane marker, indicating that the plasma membrane constituted only a small portion of the cell compared to other organelles (FIG. 15b, Cell). Analysis of the vesicle lysate showed strong bands for the cytoplasmic marker and weak bands for the plasma membrane marker, and no observable bands for other organelles (FIG. 15b, Bleb). Similarly, analysis of the B200 extrudate showed only bands for the cytoplasmic and plasma membrane markers, but the intensity of the plasma membrane marker was significantly higher than that of the cytoplasmic marker (FIG. 15b, B200). This is consistent with the relative plasma membrane content quantified by the mCherry:GFP expression ratio, indicating that the bleb-extruded nanovesicles are mainly composed of the plasma membrane of the original cell.

### Example 10

### Production and Characterization of SARS-CoV-2 Virus-Mimetic Nanovesicles

### 10-1. Production of SARS-CoV-2 Virus-Mimetic Nanovesicles

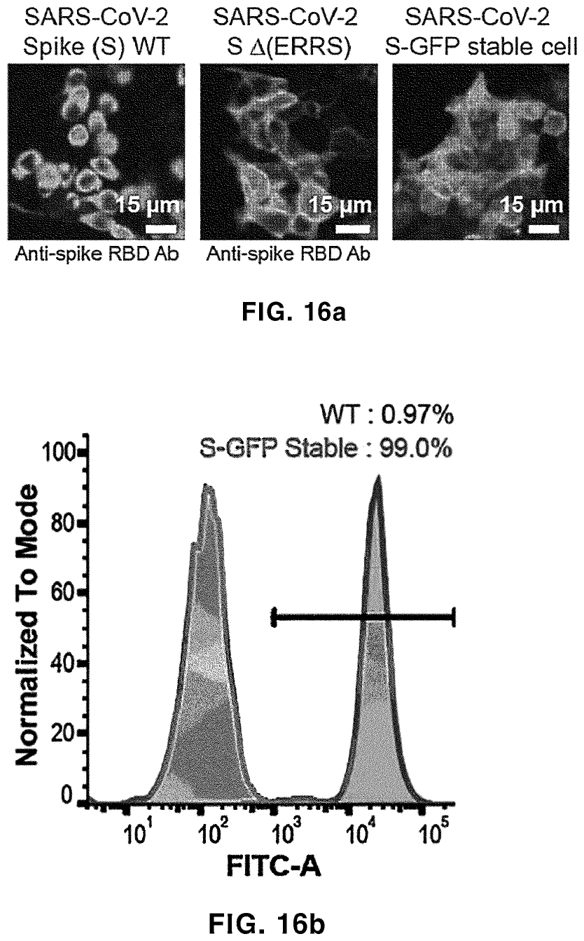

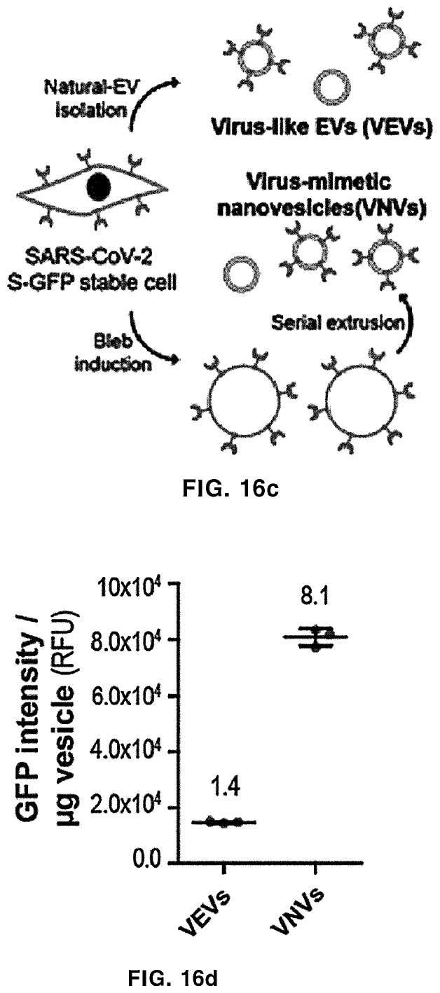

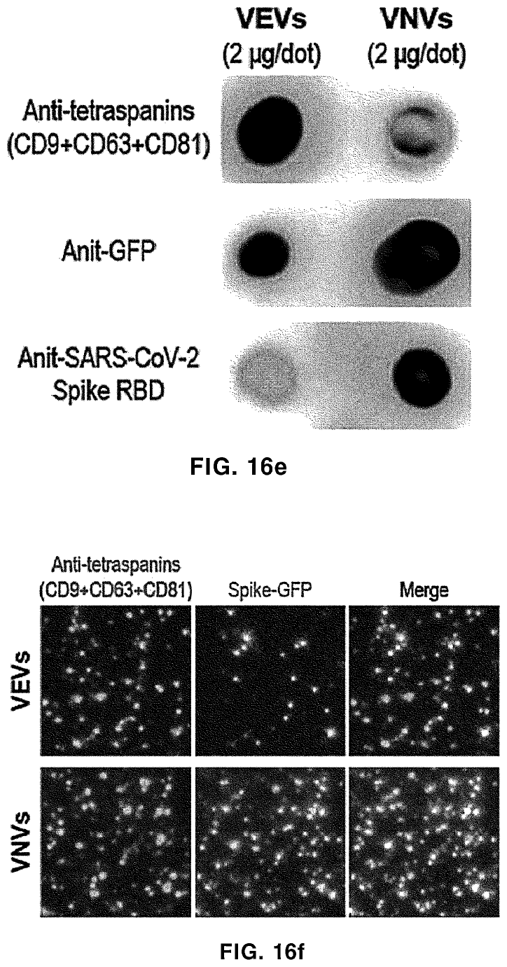

SARS-CoV-2 virus-mimetic nanovesicles were constructed by using a cell line expressing SARS-CoV-2 spike protein in the plasma membrane (FIGS. 16a to 16c).

First of all, in order to generate a stable HEK293 cell line expressing SARS-CoV-2 spike glycoprotein in the plasma membrane, transfected cells were treated with 250 μg/mL hygromycin B to select antibiotic-resistant cells. Spike-GFP-positive cells were isolated by using FACS and seeded into 96-well plates at a concentration of 1 cell per well. Afterwards, clones showing strong GFP expression in the plasma membrane were examined under an epifluorescence microscope (IX63, Olympus) and selected for further culture. The bleb induction and separation steps and the continuous extrusion steps for producing VNV were performed in the same manner as in Examples 6-2 and 6-3.

As confirmed in the left and middle fluorescence microscopy images of FIG. 16a, the endoplasmic reticulum retention signal (ERRS) located at the C-terminus of the wild-type (WT) S protein localizes the expression to the endoplasmic reticulum (ER) of the cell, and thus, the mutant S protein lacking ERRS should be used to express the S protein on the plasma membrane of the cell. In addition, as confirmed in the right fluorescence microscopy image of FIG. 16a, a stable HEK293 cell line expressing the S protein (S-GFP) with a GFP fusion at the C-terminus that may interfere with the ERRS function was generated. As confirmed in FIG. 16b, flow cytometry analysis results showed that virtually 100% of the cells successfully expressed high levels of S-GFP in the plasma membrane.

SARS-CoV-2 VNVs were generated by utilizing S-GFP cells according to the established procedure of FIG. 16c. Notably, the HEK293 S-GFP cells can naturally secrete EVs that express spike-GFP, displaying characteristics resembling the SARS-CoV-2 virus.

Accordingly, SARS-CoV-2-like EVs (VEVs) were isolated and thoroughly characterized by comparing the spike protein efficiency with SARS-CoV-2 VNV.

### 10-2. Isolation of Extracellular Vesicles

EVs were isolated by using a standard differential ultracentrifugation (DUC) method. Spike-GFP HEK293 cells were cultured until 80 to 90% confluence was reached, and then, the culture medium was replaced with DMEM without supplements. After additional 24 hours of culture, the conditioned medium was collected and centrifuged at 500×g for 10 minutes at 4° C. to remove dead cells. The supernatant was subjected to a second centrifugation step at 3,000×g for 20 minutes at 4° C. to remove cell debris. Subsequently, the medium was ultracentrifuged at 100,000×g for 2 hours at 4° C. to isolate EVs. The resulting pellet was resuspended in PBS and stored at −80° C. for additional experiments.

### 10-3. Analysis of the Size and S Protein Content of SARS-CoV-2 VNV

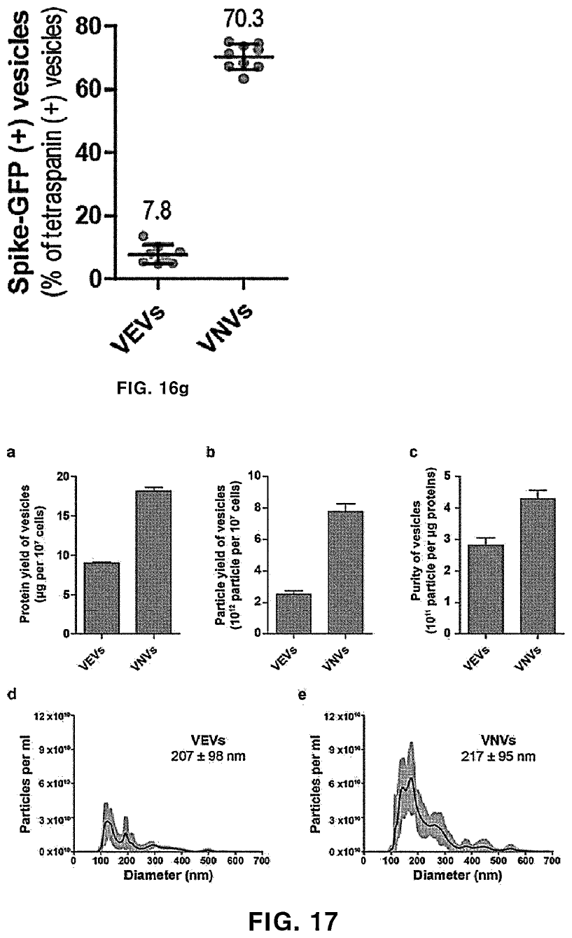

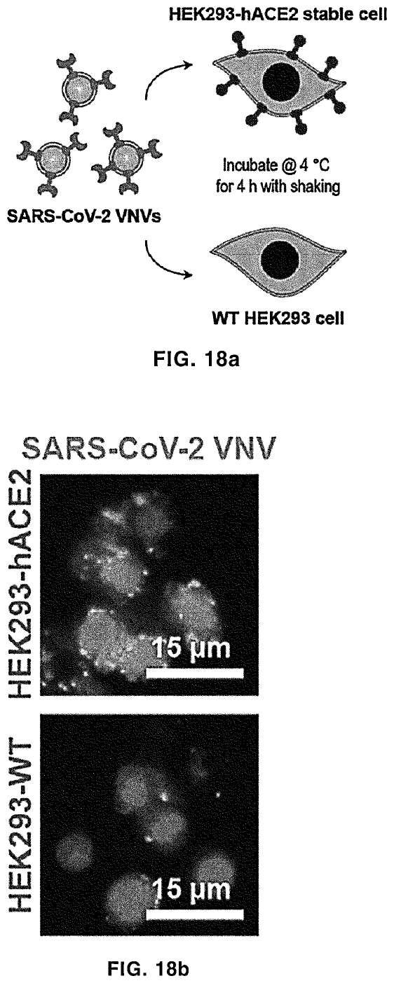

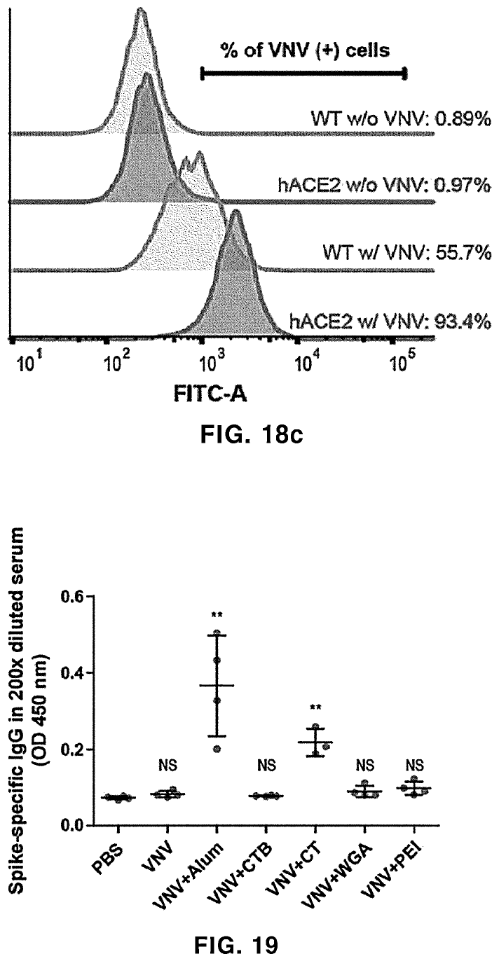

According to the NTA analysis in FIG. 17, both VEVs and VNVs had almost identical size distributions. However, the yield of VNVs considerably surpassed that of VEVs in terms of both protein quantity and particle count.

In order to determine the relative spike content per 1 μg of vesicles, the GFP intensity was measured. As a result, as confirmed in FIG. 16d, it was shown that the GFP intensity of 1 μg VNVs was approximately 5 times higher than that of 1 μg VEVs, suggesting a superior expression of the S protein in the VNVs than the VEVs.

### 10-4. Dot Blot Analysis

For dot blot analysis, the PVDF membrane was pre-wetted with methanol (Samchun, M1450), and 2 μg of protein in a 10 μL droplet was blotted onto the membrane, and then completely dried. In order to block the membrane, a solution consisting of tris-buffered saline (TBS) supplemented with 3% (w/v) bovine serum albumin (BSA) (GenDEPOT, A0100-010) and 0.05% Tween-20 (Sigma, 655204) was used at room temperature with gentle shaking. Dot-blot membranes were incubated for 2 hours at room temperature with gentle shaking with the following primary antibodies: CD9 (SCBT, SC51575), CD63 (SCBT, SC59286), CD81 (SCBT, SC23962), GFP (SCBT, SC9996) and SARS-CoV-2 (Invitrogen, PA5-114528). All antibodies were included in the blocking solution. After primary antibody incubation, the membranes were incubated with secondary antibodies conjugated with horseradish peroxidase (anti-rabbit HRP, SCBT, SC2004; anti-mouse HRP, SCBT, SC2005) at room temperature for 1 hour. Subsequently, the membranes were incubated with chemiluminescent substrate (Amersham, RPN2232), and signals were captured by using a c-Digit Western Blot Scanner (LI-COR). The bands in the cropped images were detected under the same imaging conditions.

As confirmed in FIG. 16e, the detection results of three tetraspanin EV markers (CD9, CD63, and CD81) in VNV and VEV showed that VEV exhibited higher expression of EV markers than VNV. However, the detection of S protein and S-associated GFP expression clearly indicates that VNV contains significantly more S protein than VEV. This supports that the proportion of vesicles expressing SARS-CoV-2 S protein among the total vesicles in the sample was significantly higher in VNV than in VEV.

### 10-5. Single-Vesicle Co-Localization Analysis

In order to gain insight into the expression of S protein in individual vesicles, single-vesicle co-localization analysis was performed by using total internal reflection fluorescence microscopy (TIRFm).

Single-vesicle co-localization analysis was performed by using the established method in the literature [Han, C. et al. Single-vesicle imaging and co-localization analysis for tetraspanin profiling of individual extracellular vesicles. Journal of Extracellular Vesicles 10 (3) (2021)]. Briefly, VNV and EV were biotinylated by using sulfo-NHS biotin reagent (Thermo Scientific, 21217) and immobilized on DDS-Tween-20 (DT20) passivated glass surfaces. The preparation of the DT20 surface was performed according to the method in the literature [Hua, B. et al. An improved surface passivation method for single-molecule studies. Nat Methods 11, 1233-1236 (2014)]. Surface-bound vesicles were labeled for 10 minutes with a combination of primary antibodies against CD9, CD63 and CD81 (clones: see Western Blot section, 5 μg/mL each). Subsequently, vesicles were then fluorescently labeled by using Alexa Fluor (AF) 647-conjugated anti-mouse IgG secondary antibody (Invitrogen, A-21240). Surfaces were washed three times and imaged by using TIRFm (IX73, Olympus). Spike-GFP and AF 647 tetraspanin signals located within a distance of 3 pixels (approximately 300 nm) were considered colocalized.

The red channel in FIG. 16f shows that the number of vesicles visualized by tetraspanin labeling was similar in both VEV and VEV samples. However, there was a significant difference in the number of GFP signals representing S protein-positive vesicles. In particular, as confirmed in the green channel of FIG. 16f, the VNV sample contained about 10 times more GFP-positive vesicles than the VEV sample. In addition, the single-vesicle colocalization analysis of tetraspanin and GFP signals, as confirmed in the merge of FIG. 5f and FIG. 16g, showed that about 70% of the vesicles in the VNV sample expressed S-associated GFP, whereas only about 8% of the vesicles in the VEV sample expressed S-associated protein. This means that more than 90% of VEV did not show a positive signal for the SARS-CoV-2 S protein. Overall, these results suggest that VNV is more suitable for use as enveloped virus-mimetic nanovesicles because VNV has a more uniform SARS-CoV-2 S protein expression profile and higher S-GFP content than VEV.

### Example 11

### Verification of Biological Characterization of SARS-CoV-2 Virus-Mimetic Nanovesicles

The S proteins expressed in VNVs could potentially suffer damage or modification during the production of VNVs, compromising their efficacy as vaccines. In order to ensure that the S proteins in the VNVs were intact, an analysis was performed to demonstrate the affinity between the VNVs and hACE2-expressing HEK293 cells.

SARS-CoV-2 VNV was diluted to a final concentration of 40 μg/mL VNV in complete growth medium. The VNV-containing medium was incubated with wild-type or hACE2-expressing HEK293 cell lines (4×10⁵ cells) at 4° C. for 4 hours with gentle shaking to minimize nonspecific vesicle uptake unrelated to spike-hACE2 interaction, as shown in FIG. 7a. Afterwards, the cells were then washed with PBS and fixed with 4% paraformaldehyde (PC2205-100-74, Biosesang) at room temperature for 10 minutes. Fixed cells were washed twice with PBS. Half of the cells were examined by using an epifluorescence microscope (Olympus, IX63), and the other half were analyzed by using a flow cytometer (BD, LSRFortessa).

Microscopic examination of the VNV-treated cell lines revealed a clear distinction between the two cell lines. Namely, as confirmed in FIG. 7b, hACE2-expressing HEK293 cells exhibited extensive S-GFP signals originating from the VNVs, whereas only a few WT cells displayed detectable GPF signals. In addition, the quantification results of S-GFP-positive cells using flow cytometry in FIG. 7c confirmed that nearly 100% of hACE2 cells internalized S-GFP-positive VNVs, whereas only approximately half of the WT cells took up the VNVs. These findings strongly indicate that the spike proteins of SARS-CoV-2 VNV remain functional and can efficiently bind to the hACE2 receptor, suggesting that the S proteins in VNVs are intact and capable of interacting with their target receptor.

### Example 12

### Biodistribution Analysis of Vaccines Including SASR-CoV-2 Virus-Mimetic Nanovesicles

### 12-1. Intramuscular and Intranasal Administration of SARS-CoV-2 VNV Vaccine

All experiments involving laboratory mice were conducted in accordance with the guidelines set by the Institutional Animal Care and Use Committee (IACUC) of Pohang University of Science and Technology (POSTECH-2022-0015). Female BALB/C mice (6 weeks old) were procured from a specific pathogen-free (SPF) area of the POSTECH animal facility. The mice were housed in a controlled environment with a 12-hour light:12-hour dark cycle and a constant temperature of 23° C., and were provided ad libitum access to food and water.

Different adjuvants and VNV combinations were tested to determine the optimal adjuvant for maximizing the immunogenicity of VNV. As a result, as confirmed in FIG. 19, when the same quantity of VNV was administered intramuscularly, aluminum hydroxide (alum) induced the highest level of S-specific IgG formation followed by cholera toxin (CT). However, other adjuvants such as cholera toxin R subunit (CTB), wheat germ agglutinin (WGA) and polyethyleneimine (PEI) failed to induce antibody formation at the same dose. Based on this evaluation, alum was selected as the adjuvant for IM injection, while CT was chosen for IN administration owing to the infrequent use of alum in intranasal delivery.

### 12-2. In Vivo Imaging Analysis

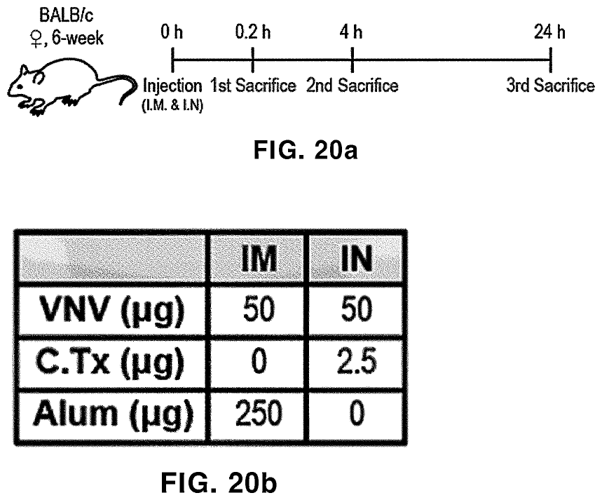

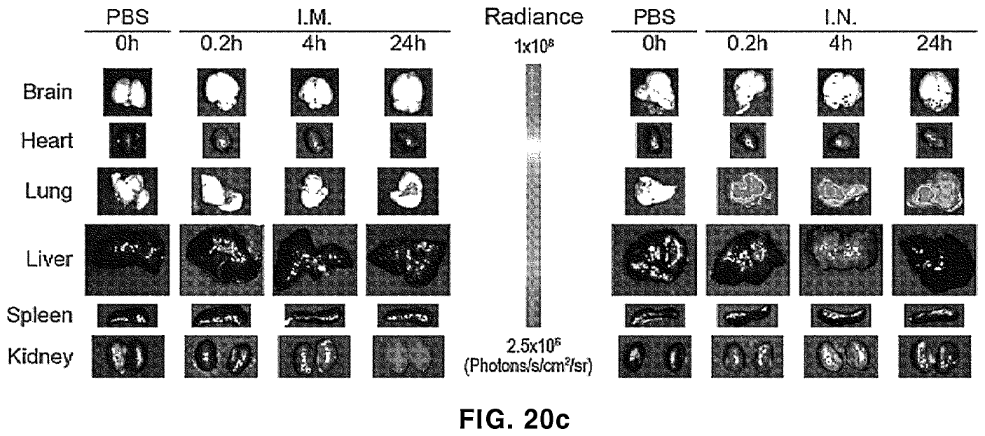

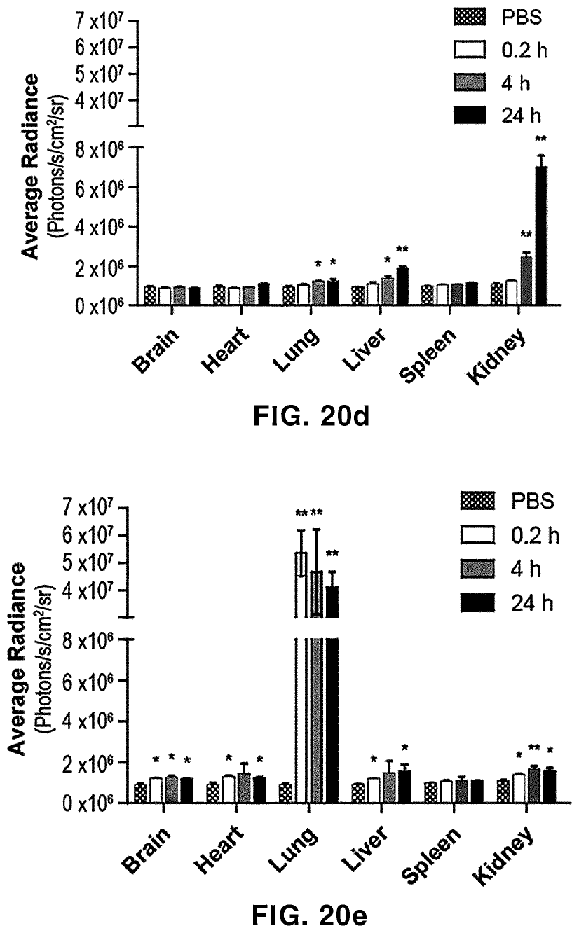

Subsequently, the in vivo biodistribution of VNV vaccines administered via IM and IN routes was examined. The in vivo biodistribution of VNV over time was evaluated by using an in vivo fluorescence imaging system. The design of the biodistribution study is shown in FIG. 20a, and the composition of the vaccine administered intramuscularly and intranasally is shown in FIG. 20b.

The animals were divided into three groups: PBS, IM and IN. The PBS group was administered with 50 μL of PBS via the intranasal route and 100 μL of PBS via the intramuscular route. The IM group was administered with 100 μL of a solution containing 50 μg of VNV and 250 μg of alum via the intramuscular route. The IN group was administered with 50 μL of a solution consisting of 50 μg of VNV and 2.5 μg of CT via the intranasal route. Before administration, VNV was labeled with cy5.5 NHS ester dye (GE, PA15601), and unbound dye was removed through two rounds of ultracentrifugation. After administration, mice were euthanized at 0.2, 4 and 24 hours, and vital organs (brain, heart, lung, liver, spleen and kidney) were isolated for fluorescence imaging to detect Cy5.5 signals from VNV.

As a result of analysis, as confirmed in FIG. 20c, VNVs administered via the IM route was filtered by the liver and kidneys through the systemic circulation 4 hours after administration. The VNV signals detected in the kidneys of the IM-injected mice notably increased 24 hours after administration, indicating active renal filtration of the IM-administered VNVs. As a result of analyzing the IN-administered mice, as confirmed in FIG. 20d, VNV signals were present only in the lungs. In contrast to IM administration, no distinct renal or hepatic filtration of VNV was observed 24 hours after IN administration. Hence, it can be inferred that the IM administration of VNVs could induce a stronger systemic immune response, while IN administration could induce a stronger local immune response.

### Example 13

### Confirmation of Systemic and Local Humoral Immune Responses Induced by SARS-CoV-2 VNV Vaccination

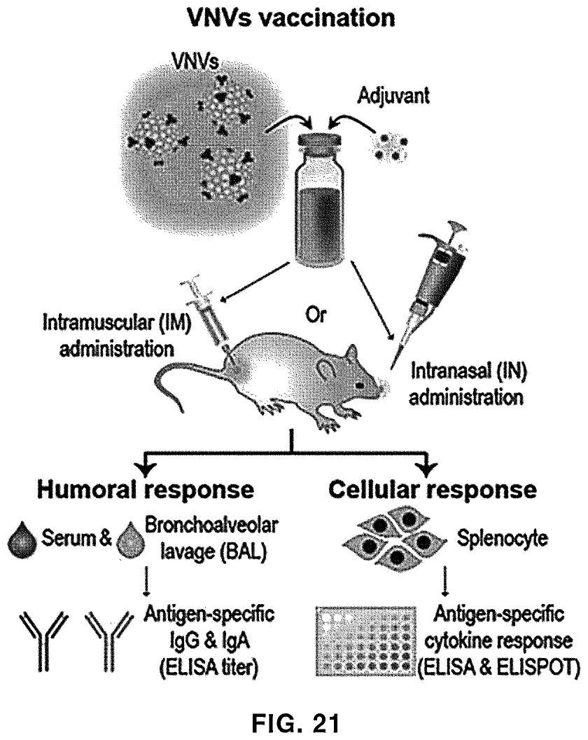

For the preclinical evaluation of vaccine efficacy, SARS-CoV-2 VNV was formulated by mixing the same with an immune enhancer, alum or cholera toxin, as shown in FIG. 21, and then administered intramuscularly or intranasally.

### 13-1. Mice Immunization

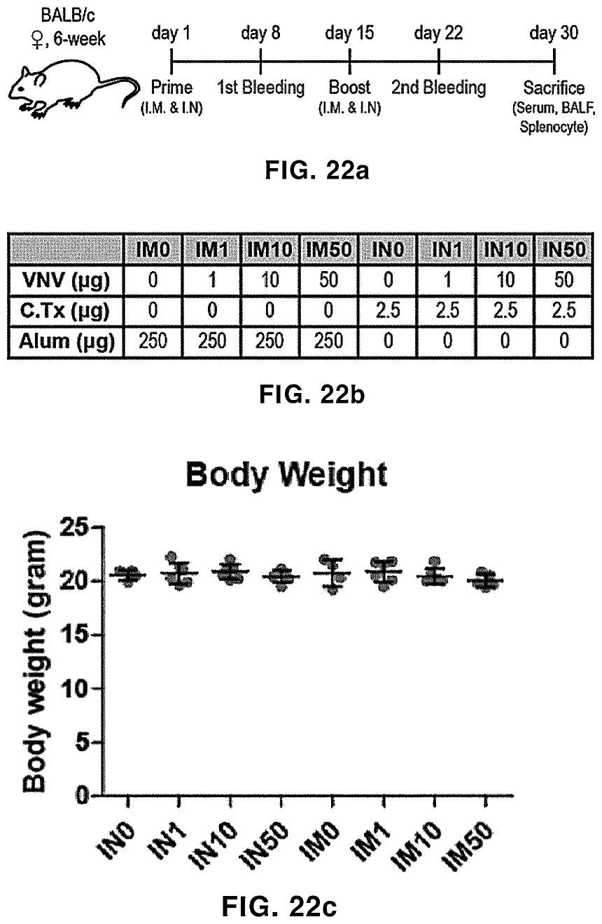

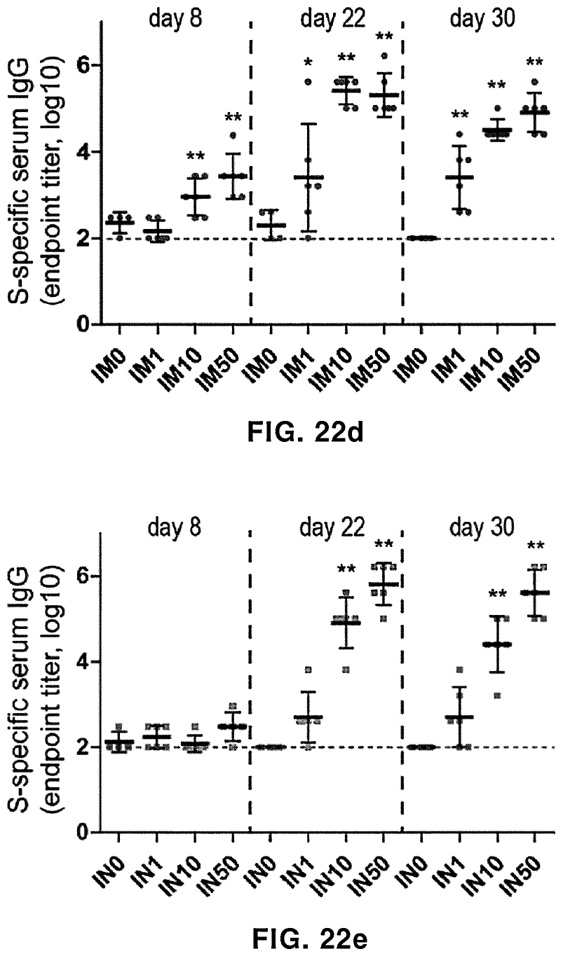

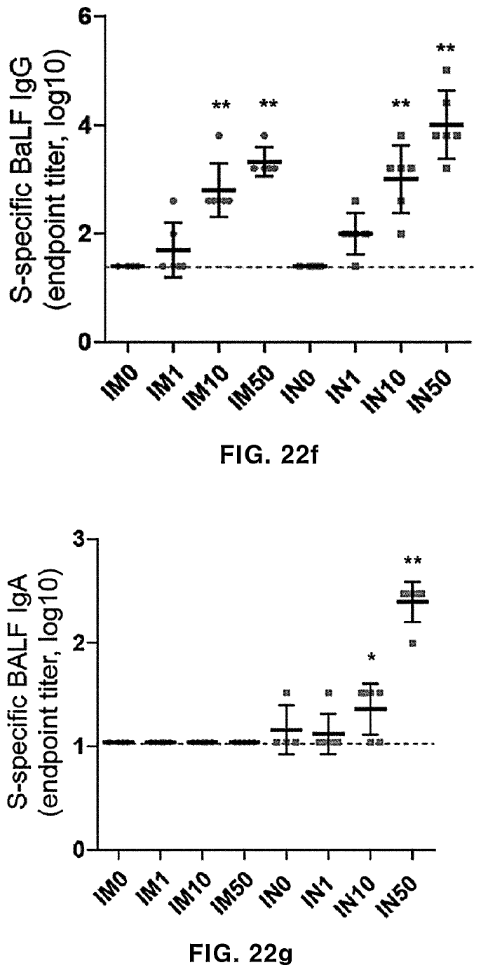

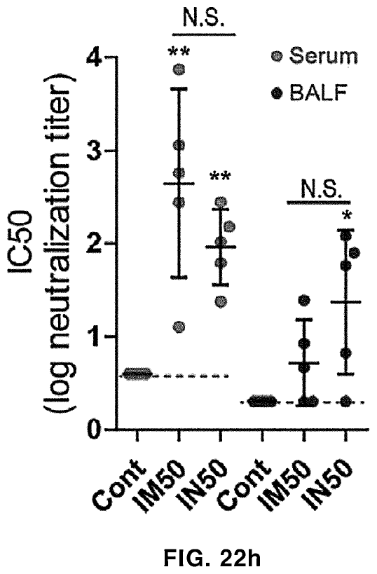

Two doses of VNV vaccine (primary and boost immunization) were administered to mice at two-week intervals to evaluate the immunogenicity of the SARS-CoV-2 VNV vaccine in a dose-dependent manner. The design of the VNV vaccination study is shown in FIG. 22a, and the compositions of the vaccines administered intramuscularly and intranasally are shown in FIG. 22b.

The animals were divided into eight groups: IM0, IM1, IM10, IM50, IN0, IN1, IN10 and IN50. The IM group was administered with 0, 1, 10 and 50 μg of VNV via the intramuscular route together with 250 μg of alum in a total dose of 100 μL. The IN group was administered with 0, 1, 10 and 50 μg of VNV via the intranasal route together with 2.5 μg of CT in a total dose of 50 μL. All animals were immunized twice at 2-week intervals. One week after immunization, a small amount of blood (less than 10 μL) was collected from the animals by tail docking. On day 30 after the initial immunization, all mice were humanely euthanized by using Avertin (2,2,2-tribromoethanol; Sigma, T48402). Blood was collected by cardiac puncture and incubated at room temperature for 1 hour to allow clotting. The blood was then centrifuged at 2,000×g for 10 minutes at 4° C., and the supernatant was carefully collected for additional analysis. The spleen was isolated and stored in cold RPMI 1640 medium (Gibco, 61870036) supplemented with 10% FBS and antibiotics. Subsequently, the spleen was then crushed on a cell strainer by using a syringe plunger. The spleen cells were centrifuged at 800×g for 3 minutes at 4° C., and the resulting pellet was resuspended in 1 mL of ACK lysis buffer (Gibco, A1049201). In order to lyse red blood cells, the cells were incubated at room temperature for 5 minutes. After incubation, the cells were centrifuged at 800×g for 3 minutes at 4° C. and used for additional analysis. BALF was collected by using 1 mL of PBS through a catheter inserted into the trachea. The collected BALF was centrifuged at 2,000×g for 10 minutes at 4° C., and the supernatant was carefully collected for additional analysis.

As shown in FIG. 22c, after administration of two doses of VNV vaccine, no mortality cases were observed in the mice, and the body weights of the vaccinated animals were similar to those of the PBS-administered animals. This indicates that all doses of the VNV vaccine were tolerated by the mice without toxicity.

### 13-2. Spike-Specific Antibody Titers

Spike-specific antibody titers in serum and BALF were quantified by using an ELISA-based method. In order to coat 96-well microtiter plates (Sigma, M9410), recombinant SARS-CoV-2 spike protein (S1+S2 ECD, Sino Biological, 40589-V08H4) was diluted to a concentration of 1 μg/ml (w/v) in PBS, and the plates were incubated overnight at 4° C. Subsequently, the plates were then blocked with 2% (w/v) BSA solution in PBS (blocking buffer) for 1 hour at room temperature and washed three times with PBS containing 0.05% Tween-20 (PBST). For serum and BALF analysis, samples isolated from mice were incubated with the coated plates for 1 hour at room temperature with gentle shaking. Serum samples were diluted 100-fold to achieve the highest concentration and then serially diluted 3-fold for both IgG and IgA titers. BALF samples were diluted 25-fold for IgG titration and 11-fold for IgA titration, and both were serially diluted accordingly. After incubation, the plates were washed three times with PBST and incubated with detection antibodies for 1 hour at room temperature with gentle shaking. Anti-mouse IgG-HRP antibody (Invitrogen, 31430) was used for IgG detection, and anti-mouse IgA-HRP antibody (Invitrogen, 62-6720) was used for IgA detection. After washing once more with PBST, the plates were treated with 100 μL of TMB substrate (GenDEPOT, #T3550-050) for 30 minutes at room temperature, and the reaction was stopped with 1 M sulfuric acid (Samchun, S1431). Signals lower than the mean and 3 times the standard deviation of the blank wells were considered undetected. The titer of each sample was determined as the reciprocal of the highest dilution that could still be detected.

As shown in FIG. 22d, serum analysis showed that mice that were administered with 10 and 50 μg of VNV via the IM route showed increased S-specific serum IgG titers after the first immunization (day 8), which were significantly increased after the second immunization (days 22 and 30). However, mice that were administered only with 1 μg of VNV via the IM route did not produce detectable levels of S-specific antibodies after the first immunization (day 8). Although the IgG titers increased after the second immunization, these titers were significantly lower than those in mice administered with the higher VNV doses (days 22 and 30).

In the case of IN administration, as confirmed in FIG. 22e, no mice were observed to produce detectable titers of S-specific serum IgG after the first immunization (day 8). After the second IN administration (days 22 and 30), mice that were administered with 10 and 50 μg of VNV showed a significant increase in the titers of S-specific serum IgG. However, mice that were administered with 1 μg of VNV via the IN route did not produce any notable titers of S-specific IgG even after the second immunization.

As confirmed in FIG. 22f, the analysis of BALF obtained on day 30 of immunization revealed that mice that were administered with 10 and 50 μg of VNV via IM or IN showed a marked increase in IgG antibody titers. However, as confirmed in FIGS. 22d to 22f, the IgG titers in BALF were lower than those in serum, and the groups that were administered with 1 μg VNVs did not demonstrate a significant elevation in BALF titers. Conversely, the analysis of IgA antibodies exhibited a distinctly different pattern compared to the IgG analysis. As confirmed in FIG. 22g, the groups that were administered with 10 and 50 μg VNVs via the IN route showed substantial increases in IgA antibody titers in BALF, whereas the IM administration groups did not show elevated titers.

### 13-3. Spike Neutralization Titer

In order to determine whether the antibodies induced by the VNV vaccine could neutralize the binding of the SARS-CoV-2 S protein to the hACE2 receptor, an ELISA-based neutralization assay was performed by using serum and BALF obtained from mice that were administered with 50 μg VNV via IM or IN on day 30. Initially, microtiter plates were prepared with 2 μg/mL coating of SARS-CoV-2 spike protein and incubated overnight at 4° C. Subsequently, blocking buffer was then applied to the plates for 2 hours at room temperature, followed by three consecutive washes with phosphate-buffered saline with Tween 20 (PBST). Afterwards, the plates were then exposed to serum and BALF for 1 hour at 37° C. For maximum sample concentration, 4-fold diluted serum and 2-fold diluted BALF were used, and serial dilutions were used for neutralization titration. After incubation, the plates were washed three times with PBST and further incubated with 400 ng/mL biotinylated recombinant hACE2 protein for 30 minutes at 37° C. After washing, the plates were exposed to HRP-avidin reagent (BioLegend, 405103) for 15 minutes at room temperature. After washing once more, TM substrate was added and reacted at room temperature for 30 minutes to detect spike-bound hACE2 protein. The reaction was stopped by using 1 M sulfuric acid, and the IC₅₀ value was calculated by using GraphPad Prism 5.

As confirmed in FIG. 22h, the sera of the IM and IN groups showed significant neutralizing ability against SARS-CoV-2, indicating that VNV vaccine-induced antibodies could provide protection (FIG. 22h, Serum). In addition, the BALF obtained from the IN group showed significantly enhanced neutralizing ability against SARS-CoV-2 infection compared to the control group administered PBS, suggesting that IN administration of the VNV vaccine could confer mucosal immunity (FIG. 22h, BALF).

### Example 14

### Confirmation of Cellular Immune Responses Induced by SARS-CoV-2 VNV Vaccination

The extent of S-specific cellular immune response to VNV vaccine was evaluated by using splenocytes isolated from all mouse groups on day 30 after immunization.

The live splenocytes were quantified by using a hemocytometer with trypan blue dye (Gibco, 15250061) and then cultured in 96-well tissue culture plates (Corning, 3598). For ELISA analysis, 2×10⁶ splenocytes in 200 μL RPMI 1640 medium supplemented with 10% FBS were stimulated with 1.25 μg/mL PepMix SARS-CoV-2 containing the entire SARS-CoV-2 spike glycoprotein sequence (JPT, PM-WCPV-S-1). After 24 hours of stimulation, the supernatant was collected, centrifuged at 800×g for 3 minutes at 4° C. to remove cells, and then used for ELISA analysis. For peptide-stimulated specific cytokine quantification, the mouse interferon-gamma (IFN-γ) ELISA kit (Biolegend, 430807) and the mouse interleukin-2 (IL-2) ELISA kit (Biolegend, 431007) were used.

For the ELISPOT analysis, 1×10⁶ splenocytes were added to plates that were pre-coated with mouse IFN-γ antibody (MABTECH, 3321-4AST-2) in 200 μL RPMI 1640 medium supplemented with 10% FBS, and stimulated with 1.25 μg/mL PepMix SARS-CoV-2 for 16 hours. Cells specifically responding to spike stimulation were identified by using antibodies and alkaline phosphatase (ALP)-conjugated streptavidin according to the manufacturer's instructions (MABTECH, 3321-4AST-2).

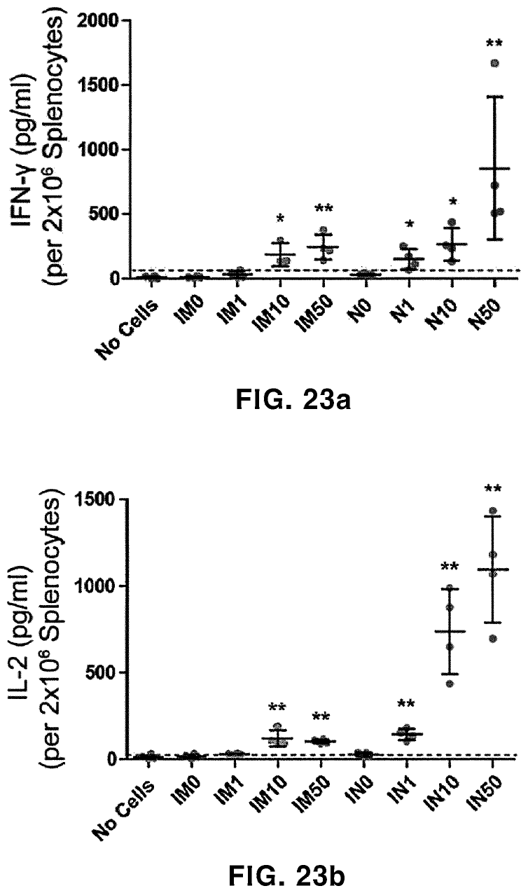

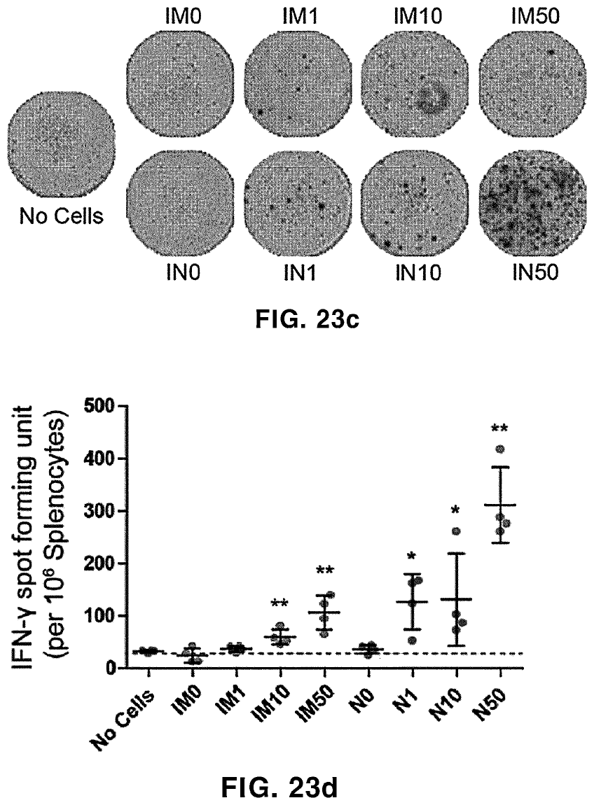

As confirmed in FIGS. 23a and 23b, splenocytes from mice that were administered with 10 and 50 μg VNV via the IM or IN route showed significantly increased levels of proinflammatory cytokines, that is, IFN-μ and IL-2, when stimulated with a mixture of peptides derived from the receptor binding domain (RBD) of the S protein. In addition, as confirmed in FIGS. 23c and 23d, ELISPOT analysis of splenocytes identified by S-RBD stimulation showed that mice that were administered with 50 μg VNV via the IM or IN route had a higher number of immune cells that were capable of recognizing viral proteins and producing proinflammatory cytokines to protect the host from infection, indicating an enhanced cellular immune response. In particular, the degree of cellular immune response was higher in the IN administration group than in the IM administration group.

Based on the above examples, it was demonstrated that the vaccine composition utilizing the virus-mimetic nanovesicles of the present invention can induce a cellular immune response that generates virus-specific immune cells in addition to a humoral immune response through antibody production.

This patent application was supported by the National Research Foundation of Korea (1711137918, (Participation 3) Development of an integrated platform for full-cycle diagnostic liquid biopsy for customized treatment of prostate cancer) funded by the Government of the Republic of Korea (multi-ministerial) in 2024 and by the National Research Foundation of Korea (1345352500, Development of an eVLP vaccine platform utilizing bio-derived nanoparticle analysis technology) funded by the Government of the Republic of Korea (Ministry of Education) in 2024.

1. A method for preparing nanovesicles which express an immunomodulatory protein or targeting protein, comprising the steps of:
(a) establishing a cell line that expresses an immunomodulatory protein or targeting protein inside and outside the plasma membrane;
(b) treating a bleb-inducing agent to a culture medium comprising the cell line to induce and separate plasma membrane blebs; and
(c) reducing the size of the blebs to generate and separate nanovesicles.

2. The method of claim 1, wherein the immunomodulatory protein of step (a) comprises at least one selected from the group consisting of (i) to (iv) below:
(i) a pathogenic antigen;
(ii) a signal transduction membrane protein or adaptive immune cell membrane protein marker which is present on the cell membrane and immunochemically involved in adaptive immunity;
(iii) a signal transduction membrane protein or innate immune cell membrane protein marker which is present on the cell membrane and immunochemically involved in innate immunity; and
(iv) a signal transduction membrane protein or membrane protein marker which is expressed on the surface of a tumor.

3. The method of claim 2, wherein the pathogenic antigen is a virus-specific antigen, a bacteria-specific antigen, a parasite-specific antigen or a disease-related human antigen.

4. The method of claim 1, wherein the targeting protein is a membrane protein which is expressed in the tissue or cell to be targeted or a protein that binds thereto.

5. The method of claim 1, wherein the cell line of step (a) is a single antigen-expressing cell line which expresses one virus-specific antigen or a multi-antigen-expressing cell line which expresses one virus-specific antigen and additional other pathogenic antigens or additional other proteins,
wherein the additional pathogenic antigen is a virus-specific antigen, bacteria-specific antigen, parasite-specific antigen, disease-related human antigen or innate immune stimulating antigen which is different from the virus-specific antigen of step (a), and
wherein the additional other protein is at least one selected from the group consisting of a receptor, a ligand and an antibody.

6. The method of claim 1, wherein the bleb-inducing agent of step (b) is treated for 2 to 5 hours.

7. The method of claim 1, wherein the bleb-inducing agent of step (b) is N-ethyl maleimide.

8. The method of claim 1, wherein the method for reducing the size of the blebs in step (c) is at least one method selected from the group consisting of a porous filter extrusion method, an ultrasonic treatment method, a micro-nozzle passage method, a micro-fluid chip passage method and a spray method.

9. The method of claim 1, further comprising the step of:
(d) purifying nanovesicles.

10. The method of claim 9, wherein the step of purifying nanovesicles is performed by a differential centrifugation method or a density gradient centrifugation method.

11. Nanovesicles which express an immunomodulatory protein or targeting protein, prepared by the method of claim 1.

12. The nanovesicles of claim 11, wherein when the immunomodulatory protein is a virus-specific antigen, the nanovesicles are prepared as plasma membrane bleb-based enveloped virus-mimetic nanovesicles.

13. The nanovesicles of claim 12, wherein the plasma membrane bleb-based enveloped virus-mimetic nanovesicles express the SARS-CoV-2 Spike protein.

14. The nanovesicles of claim 12, wherein the plasma membrane bleb-based enveloped virus-mimetic nanovesicles are multivalent virus-mimetic nanovesicles which express multiple antigens.

15. The nanovesicles of claim 11, wherein the immunomodulatory protein is CD80, and the targeting protein is a fusion protein comprising an antibody.

16. A vaccine composition, comprising the nanovesicles of claim 12.

17. A drug delivery vehicle, comprising the nanovesicles of claim 15.

18. A pharmaceutical composition for inducing an immune response, comprising the nanovesicles of claim 15.

19. A method for inducing immunity, comprising the step of administering the nanovesicles of claim 11 to a subject in need thereof.

20. A method for signaling or targeting in a subject, comprising the step of administering to the nanovesicles of claim 11 to a subject in need thereof.


SEQUENCE LISTING (3 sequences)

SEQ ID NO: 1 - DNA, 1596 bp, synthetic construct
  source 1..1596: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtagggggtggaggctctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagaagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggctaacttcaaggttcgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 2 - DNA, 1650 bp, synthetic construct
  source 1..1650: mol_type = other DNA
atggcttgcaattgtcagttgatgcaggatacaccactcctcaagtttccatgtccaaggctcattcttctctttgtgctgctgattcgtctttcacaagtgtcttcagatgttgatgaacaactgtccaagtcagtgaaagataaggtattgctgccttgccgttacaactctcctcatgaagatgagtctgaagaccgaatctactggcaaaaacatgacaaagtggtgctgtctgtcattgctgggaaactaaaagtgtggcccgagtataagaaccggactttatatgacaacactacctactctcttatcatcctgggcctggtcctttcagaccggggcacatacagctgtgtcgttcaaaagaaggaaagaggaacgtatgaagttaaacacttggctttagtaaagttgtccatcaaagctgacttctctacccccaacataaccgagtctggaaacccatctgcagacactaaaaggattacctgctttgcttccgggggtttcccaaagcctcgcttctcttggttggaaaatggaagagaattacctggcatcaatacgacaatttcccaggatcctgaatctgaattgtacaccattagtagccaactagatttcaatacgactcgcaaccacaccattaagtgtctcattaaatatggagatgctcacgtgtcagaggacttcacctgggaaaaacccccagaagaccctcctgatagcaagaacacacttgtgctctttggggcaggattcggcgcagtaataacagtcgtcgtcatcgttgtcatcatcaaatgcttctgtaagcacagaagctgtttcagaagaaatgaggcaagcagagaaacaaacaacagccttaccttcgggcctgaagaagcattagctgaacagaccgtcttccttgggggtggaggctctgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagaagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggctaacttcaaggttcgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 648 bp, synthetic construct
  source 1..648: mol_type = other DNA
ggtaccgccacaatgcggctgcccgcccagctgctggggctgctgatgctgtgggtgcccggcagcagcggctacccctacgatgtgcccgactacgccgggggcggcgggtcccaggtgcagctgcaggagagcgggggcggcagcgtgcaggccggcgggagcctgaggctgagctgcacagccccagggtacaccgacagcaactactacatgagctggttcaggcaggcccctgggaaggagcgggagtgggtggccggggtgaacacaggcaggggcagcaccagctacgccgacaccgtgaaggggcggttcaccatcagccaggataacgccaagaacaccatgttcctgcagatgaacagcctgaaacccgaggacacagccatctactactgcgccgtggccgcctgccacttctgcgactcccttcccaagggacaggacgagcagatcctgtgggggcagggcactcaggtcacagtgagcagcgggggcggcgggagcggggggggcgggagcctgtgggtcatcctgttgagcgcatttgctggactgttgttgttgatgttgcttattttggctttgtggggagggggagggagcgagcagaagctgatctccgaggaggatttgtaatctaga